(19) United States
(12) Patent Application Publication
(10) Pub. No.: US 2026/0183214 A1
(43) Pub. Date: Jul. 2, 2026

(54) COMPOSITION FOR MITIGATING SUN DAMAGE, SUNBURN, OR BOTH

(71) Applicant: Sol Sciences Inc., Pasadena, CA (US)

(72) Inventors: Teo Soleymani, La Canada Flintridge, CA (US); Michael Abrouk, Miami, FL (US); Ashwinn Krishnaswamy, New York, NY (US)

(21) Appl. No.: 19/434,816

(22) Filed: Dec. 29, 2025

Related U.S. Application Data
(60) Provisional application No. 63/739,917, filed on Dec. 30, 2024.

(51) Int. Cl. A61K 8/67; A61K 8/34; A61K 8/35; A61K 8/49; A61K 8/64; A61K 8/65; A61K 8/73; A61K 8/9741; A61Q 19/00

(52) U.S. Cl. CPC A61K 8/675; A61K 8/345; A61K 8/35; A61K 8/49; A61K 8/64; A61K 8/65; A61K 8/676; A61K 8/735; A61K 8/9741; A61Q 19/00; A61K 2800/30

(57) ABSTRACT

In some aspects, the disclosure relates to a composition including niacinamide and polypodium leucotomos extract, optionally with additional photoprotective ingredients including ascorbic acid, optionally biotin, astaxanthin, bovine collagen peptides, L-glutathione, hyaluronic acid, and inositol. The composition provides rapid and measurable photoprotection, reducing UV-induced erythema by approximately 60-68% when administered 1-2 hours prior to UV exposure. Clinical studies demonstrate significant increases in minimal erythema dose (MED) with effects persisting for at least 24 hours following a single oral administration. The composition exhibits excellent formulation characteristics including superior mixability, clarity, and palatability when formulated as a beverage powder.

### CROSS-REFERENCE TO RELATED APPLICATION

This application claims the benefit of U.S. Provisional Ser. No. 63/739,917 , filed Dec. 30, 2024, which is incorporated herein by reference in its entirety.

### BACKGROUND

Environmental aging and sun damage can occur through multiple pathways that affect skin health and appearance. These pathways include exposure to ultraviolet (UV) radiation, visible light, infrared radiation, pollution, and dietary factors. UV radiation can trigger oxidative stress, DNA damage, inflammation, and degradation of structural proteins in the skin. Further, visible light and heat exposure may lead to additional oxidative damage and inflammatory responses. In addition, environmental pollutants like particulate matter, heavy metals, and ozone can compromise the skin barrier and induce oxidative cascades.

### SUMMARY

Environmental exposure to ultraviolet (UV) radiation, visible light, infrared radiation, and pollution leads to significant sun damage and accelerated skin aging, manifesting as erythema, DNA damage, oxidative stress, and degradation of skin structure. Existing topical photoprotective strategies, such as sunscreens, are limited by incomplete coverage, user compliance issues, and the need for frequent reapplication, resulting in suboptimal protection. There remains a need for an effective, convenient, and comprehensive approach to mitigate sun damage and sunburn.

The present disclosure provides a novel oral composition comprising niacinamide and polypodium leucotomos root/stem extract, optionally with additional photoprotective ingredients, which reduce the incidence and severity of sun damage and sunburn. The composition offers rapid and measurable photoprotection, as demonstrated by significant increases in minimal erythema dose (MED) and reductions in UV-induced erythema compared to baseline measurements taken prior to supplementation. This systemic approach overcomes the limitations of topical products by providing consistent, whole-body protection, improved user convenience, and enhanced efficacy against environmental skin damage.

The compositions described herein provide unexpected and superior results that were not predictable. Clinical testing demonstrated reduction of UV-induced erythema by approximately 60-75% at 2 hours post-ingestion, significantly exceeding the photoprotective effects observed with individual ingredients or conventional formulations. The composition provides effective protection even at high narrowband UVB doses (350 mJ/cm²), a level at which prior art oral photoprotectants typically show minimal efficacy. The minimal erythema dose (MED) increased substantially compared to baseline measurements, with some subjects showing improvements of up to 75% at 24 hours post-exposure. The rapid onset of photoprotection, measurable within 1-3 hours of oral administration, was not predicted from prior art teachings on niacinamide or polypodium leucotomos extract alone, both of which have historically required chronic dosing regimens to demonstrate protective effects.

The combination of niacinamide, polypodium leucotomos extract, astaxanthin, and L-glutathione produced dramatically enhanced photoprotection compared to formulations lacking any one of these components. Formulation variants containing turmeric (at concentrations from 250 mg to 1000 mg) exhibited severely impaired performance characteristics, demonstrating that not all combinations of putatively beneficial ingredients produce acceptable results. The composition demonstrated efficacy across Fitzpatrick skin phototypes II-IV, indicating broad demographic validity and clinical utility across diverse populations with varying baseline UV sensitivity.

Various aspects according to the present disclosure relate to a composition (e.g., an oral composition), which may reduce the incidence or severity of sun damage, sun burn, or both, compared to a subject who does not receive the compositions of the various embodiments described herein. Sun damage may include skin cancer, damage from free radicals, environmental aging, or combinations thereof. As mentioned above, environmental aging may be caused or triggered by exposure to sun, pollution, diet, and visible light.

In some aspects, the disclosure relates to a composition including: niacinamide; and polypodium leucotomos extract.

In some aspects, the disclosure relates to a composition including: ascorbic acid in a range of from about 7 wt % to about 15 wt % of the compositions of the various embodiments described herein; niacinamide in a range of from about 35 wt % to about 40 wt % of the compositions of the various embodiments described herein; astaxanthin in a range of from about 0.0005 wt % to about 1 wt % of the compositions of the various embodiments described herein; bovine collagen peptides in a range of from about 2 wt % to about 4 wt % of the compositions of the various embodiments described herein; L-glutathione in a range of from about 0.009 wt % to about 0.03 wt % of the compositions of the various embodiments described herein; hyaluronic acid in a range of from about 0.008 wt % to about 0.02 wt% of the compositions of the various embodiments described herein; inositol in a range of from about 35 wt % to about 40 wt % of the compositions of the various embodiments described herein; and polypodium leucotomos extract in a range of from about 6 wt % to about 15 wt % of the compositions of the various embodiments described herein.

### DETAILED DESCRIPTION

Reference will now be made in detail to certain aspects of the disclosed subject matter. While the disclosed subject matter will be described in conjunction with the enumerated claims, it will be understood that the exemplified subject matter is not intended to limit the claims to the disclosed subject matter.

Reference will now be made in detail to certain examples of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section.

In the methods described herein, the acts can be carried out in any order without departing from the principles of the disclosure, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range, and includes the exact stated value or range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the compositions of the various embodiments described herein including the material, such that about 0 wt % to about 5 wt % of the compositions of the various embodiments described herein is the material, or about 0 wt % to about 1 wt %, or about 5 wt % or less, or less than or equal to about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

Various aspects according to the present disclosure relate to a composition, which may reduce the incidence or severity of sun damage, sun burn, or both, compared to a subject who does not receive the compositions of the various embodiments described herein. Sun damage may inlcude, skin cancer, damage from free radicals, environmental aging, or combinations thereof. Environmental aging may be caused or triggered by exposure to sun, pollution, diet, and visible light.

Ultraviolet (UV) radiation-induced damage includes UVA-mediated oxidative stress and free radical generation, UVB-induced DNA damage and inflammation, UV-activated matrix metalloproteinases that degrade collagen, and UV-induced reduction in skin's natural antioxidant levels. Visible light and infrared radiation effects include blue light-induced oxidative stress, heat-induced inflammation and matrix degradation, and infrared-activated stress response pathways. Pollution-related damage includes particulate matter-induced oxidative stress, heavy metal-activated inflammatory cascades, ozone-mediated lipid peroxidation, and air pollutant-triggered barrier dysfunction. Dietary and metabolic stress includes advanced glycation end-product formation, cellular oxidative stress from processed foods, inflammation from dietary imbalances, and nutrient deficiency-related skin barrier impairment.

The compositions of the various embodiments described herein provides mitigation against these environmental aging mechanisms through free radical scavenging and antioxidant activity, DNA photoprotection and repair support, anti-inflammatory pathway modulation, cellular stress response optimization, barrier function enhancement, matrix protein preservation, and natural defense system strengthening.

The protective effects result in reduced appearance of fine lines and wrinkles, hyperpigmentation and age spots, skin texture irregularities, loss of elasticity and firmness, dehydration and barrier compromise, inflammation and redness, uneven skin tone, and photoaging markers.

The compositions of the various embodiments described herein includes, at a minimum, niacinamide (also known as nicotinamide) and polypodium leucotomos extract (also known as calaguala) the extract of polypodium leuctomos as described herein may be from the root, stem, leaf or a mixture of at least two thereof. In some examples, a polypodium leuctomos leaf extract can be extracted with water and a polypodium leucotomos root and stem extract can be extracted with a mixture of water and ethanol. Polypodium leucotomos root/stem extract is derived from the tropical fern Polypodium leucotomos, which is native to Central and South America, The plant includes polyphenolic compounds, including ferulic acid, caffeic acid, coumaric acid, and vanillic acid, which contribute to its potent antioxidant and photoprotective properties. The polypodium leuctomos extract can be obtained through aqueous or hydroalcoholic extraction processes applied to the root, stem, or whole plant material. Extracts can vary in purity depending on the method of extraction and subsequent processing. Purities of polypodium leucotomos extract can be expressed in terms of the concentration of active polyphenols or as a standardized extract ratio. For example, extracts may be standardized to contain at least 10% polyphenols, or may be described as a 10:1 extract, indicating that ten parts of raw plant material yield one part of concentrated extract. Higher purities, such as 20:1 extracts or those containing up to 20% polyphenols, can be obtained. The choice of purity and source can influence the distribution and concentration of bioactive compounds within the extract, which in turn may affect the efficacy and organoleptic properties of the final composition. Extracts sourced from wild-grown versus cultivated plants, or those processed using advanced purification techniques, may offer different profiles of phenolic constituents and antioxidant capacity. In the context of the present disclosure, polypodium leucotomos root/stem extract may be utilized in any suitable purity, including but not limited to 10:1, 20:1, or standardized polyphenol concentrations ranging from 10% to 20%, provided that the extract delivers the desired photoprotective effects and meets formulation requirements for stability and palatability.

As demonstrated herein niacinamide and polypodium leucotomos extract, work toghether to provide rapid and measurable photoprotection as demonstrated, at least, further in the Examples disclosed here.

In controlled studies involving narrowband UVB (nbUVB) exposure, compositions containing both niacinamide and polypodium leucotomos demonstrated significant reduction in acute erythema response within hours of oral administration, suggesting enhanced DNA repair mechanisms, improved cellular stress response, and potent anti-inflammatory effects.

The niacinamide can be present in a range of from about 25 wt % to about 45 wt % of the compositions of the various embodiments described herein, about 35 wt % to about 40 wt %, less than, equal to, or greater than about 25 wt %, 26, 27, 28, 29, 30, 31, 32, 33, 34, 35, 36, 37, 38, 39, 40, 41, 42, 43, 44, or about 45 wt % of the compositions of the various embodiments described herein. The niacinamide can be present in the compositions of the various embodiments described herein in a range of from about 100 mg to about 3000 mg, about 500 mg to about 1000 mg, or specifically about 1000 mg per serving. “Serving,” as used herein, refers to a single, unit dose of the compositions of the various embodiments described herein as defined by the amounts of active ingredients specified (e.g., 1000 mg niacinamide, 240 mg polypodium leucotomos extract, etc.), which may be provided in a single form (such as a powder, tablet, capsule, or beverage) or divided among multiple units intended to be administered together. When the compositions of the various embodiments described herein is provided as a powder or dry mix, a serving is the amount intended to be reconstituted in a specified volume of liquid (e.g., 200-300 mL) and consumed at one time. The serving size may be adjusted or divided across multiple units (e.g., two capsules, tablets, or scoops) to facilitate flexible dosing, provided that the total administered quantity corresponds to the defined serving

In some examples, a dose of approximately 1000 mg of niacinamide has been shown to provide measurable photoprotective effects within one to two hours of oral administration.

While not wishing to be bound by any specific theory, niacinamide is believed to function through multiple mechanisms including enhancement of DNA repair pathways, reduction of UV-induced immunosuppression, preservation of cellular energy metabolism, and support of the skin barrier function. In some particular examples, niacinamide at doses of about 500 mg to about 1500 mg can reduce the severity of UV-induced erythema by approximately 10% to 75%, 20% to 70%, 30% to 60%, less than, equal to, or greater than about 10%, 15, 20, 25, 30, 35, 40, 45, 50, 55, 60, 65, 70, or 75% compared to baseline measurements taken prior to supplementation. “Compared to baseline measurements taken prior to supplementation,” as used herein, refers to the evaluation of a subject's condition or response (such as erythema severity, minimal erythema dose, or other relevant endpoints) after administration of the compositions of the various embodiments described herein, relative to the measurements of the same parameter(s) obtained from the subject before any supplementation was given. The baseline measurement serves as the reference point for assessing the effect of the compositions of the various embodiments described herein.

The polypodium leucotomos extract can be present in a range of from about 5 wt % to about 20 wt % of the compositions of the various embodiments described herein, about 6 wt % to about 15 wt %, less than, equal to, or greater than about 5, 6, 7, 8, 9, 10, 11, 12, 13, 14, 15, 16, 17, 18, 19, or about 20 wt% of the compositions of the various embodiments described herein. The polypodium leucotomos extract can be present in a range of from about 50 mg to about 1500 mg of the compositions of the various embodiments described herein, about 230 mg to about 350 mg, or specifically about 240 mg, 480 mg, or 720 mg per serving.

In some examples, higher concentrations (e.g., 480 mg to 720 mg) may provide enhanced photoprotective effects; however, they can negatively impact organoleptic properties, particularly causing a bitter aftertaste and reduced palatability. In some examples, a concentration of about 240 mg has been identified as providing a suitable balance between clinical efficacy and acceptable taste profile, with formulations containing this amount demonstrating superior mixability, clarity, and consumer acceptability.

The polypodium leucotomos extract provides photoprotection through multiple pathways including antioxidant activity, prevention of UV-induced DNA damage, inhibition of matrix metalloproteinases, and modulation of inflammatory cascades.

In some aspects, the ratio of niacinamide to polypodium leucotomos extract ranges from about 2:1 to about 6:1 by weight, including all individual values and sub-ranges within that range, such as from about 2.5:1 to about 5.5:1, from about 3:1 to about 5:1, from about 3.5:1 to about 4.5:1, from about 4:1 to about 4.5:1, or specifically about 2:1, 2.5:1, 3:1, 3.5:1, 4:1, 4.25:1, 4.5:1, 5:1, 5.5:1, or about 6:1 by weight. The ratio of 4.5:1, as an example, has been demonstrated, in some examples, to provide optimal photoprotective effects while maintaining excellent formulation stability and palatability.

The compositions of the various embodiments described herein can further include a polyhydroxy compound. Examples of polyhydroxy compounds include, but are not limited to, ascorbic acid (vitamin C), dehydroascorbic acid, ascorbyl palmitate, sodium ascorbate, calcium ascorbate, magnesium ascorbate, potassium ascorbate, zinc ascorbate, ascorbyl glucoside, glycerin, propylene glycol, butylene glycol, pentylene glycol, sorbitol, xylitol, mannitol, and combinations thereof. The polyhydroxy compound can be present in a range of from about 5 wt % to about 20 wt % of the compositions of the various embodiments described herein, about 7 wt % to about 15 wt %, less than, equal to, or greater than about 5 wt %, 6, 7, 8, 9, 10, 11, 12, 13, 14, 15, 16, 17, 18, 19, or about 20 wt % of the compositions of the various embodiments described herein. The polyhydroxy compound can be present in a range of from about 100 mg to about 1000 mg, about 240 mg to about 280 mg, or specifically about 250 mg, less than, equal to, or greater than about 100 mg, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or about 1000 mg.

Ascorbic acid can function as an antioxidant that scavenges reactive oxygen species generated by UV exposure and supports collagen synthesis. In combination with niacinamide and polypodium leucotomos, ascorbic acid can enhance the overall photoprotective capacity of the compositions of the various embodiments described herein by providing complementary mechanisms of protection.

The compositions of the various embodiments described herein may include heterocyclic compounds such as biotin (vitamin B7), thiamine (vitamin B1), riboflavin (vitamin B2), niacin (vitamin B3), pantothenic acid (vitamin B5), pyridoxine (vitamin B6), folate (vitamin B9), cyanocobalamin (vitamin B12), pyridoxal phosphate, thiamine pyrophosphate, flavin mononucleotide, flavin adenine dinucleotide, and combinations thereof. Heterocyclic compounsd may be present in a range of from about 0.05 wt % to about 5 wt % of the compositions of the various embodiments described herein, about 0.09 wt% to about 2 wt %, less than, equal to, or greater than about 0.05 wt %, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.5, 3.0, 3.5, 4.0, 4.5, or about 5 wt % of the compositions of the various embodiments described herein. In terms of amount per serving or unit dose, the heterocyclic compound may be present in a range of from about 0.50 mg to about 10 mg, about 3 mg to about 7 mg, or specifically about 5 mg, less than, equal to, or greater than about 0.50 mg, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, or about 10 mg. Biotin can support cellular metabolism and energy production, which are critical for maintaining skin barrier function and supporting DNA repair processes that may be compromised by UV exposure.

The compositions of the various embodiments described herein may include carotenoid compounds such as astaxanthin, beta-carotene, alpha-carotene, lycopene, lutein, zeaxanthin, beta-cryptoxanthin, canthaxanthin, fucoxanthin, capsanthin, capsorubin, and combinations thereof. The carotenoid compound may be present in a range of from about 0.0005 wt% to about 1 wt % of the compositions of the various embodiments described herein, about 0.009 wt % to about 0.005 wt %, less than, equal to, or greater than about 0.0005 wt %, 0.001, 0.002, 0.003, 0.004, 0.005, 0.006, 0.007, 0.008, 0.009, 0.01, 0.02, 0.03, 0.04, 0.05, 0.1, 0.2, 0.3, 0.4, 0.5, 0.6, 0.7, 0.8, 0.9, or about 1 wt % of the compositions of the various embodiments described herein. In terms of amount per serving or unit dose, the carotenoid compound may be present in a range of from about 0.5 mg to about 20 mg, about 2 mg to about 6 mg, or specifically about 4 mg, less than, equal to, or greater than about 0.5 mg, 1.0, 1.5, 2.0, 2.5, 3.0, 3.5, 4.0, 4.5, 5.0, 6.0, 7.0, 8.0, 9.0, 10.0, 12.0, 14.0, 16.0, 18.0, or about 20 mg.

Astaxanthin provides powerful antioxidant protection and has been shown to accumulate in skin tissues where it can directly quench singlet oxygen and other reactive species generated by UV exposure. In some examples, astaxanthin at concentrations of about 4 mg per serving provides optimal effects without negatively impacting formulation clarity or stability. Higher concentrations (e.g., 6 mg) may result in increased turbidity and reduced mixability in beverage formulations.

The compositions of the various embodiments described herein may include polypeptide compounds such as bovine collagen peptides, marine collagen peptides, porcine collagen peptides, chicken collagen peptides, hydrolyzed collagen, collagen tripeptides, collagen dipeptides, elastin peptides, keratin peptides, silk peptides, soy peptides, wheat peptides, and combinations thereof. The polypeptide compound may be present in a range of from about 0.5 wt % to about 15 wt % of the compositions of the various embodiments described herein, about 2 wt % to about 4 wt %, or about 2 wt % to about 12 wt %, less than, equal to, or greater than about 0.5 wt %, 1.0, 1.5, 2.0, 2.5, 3.0, 3.5, 4.0, 4.5, 5.0, 6.0, 7.0, 8.0, 9.0, 10.0, 11.0, 12.0, 13.0, 14.0, or about 15 wt % of the compositions of the various embodiments described herein. In terms of amount per serving or unit dose, the polypeptide compound may be present in a range of from about 10 mg to about 1000 mg, about 80 mg to about 120 mg, or about 100 mg to about 500 mg, less than, equal to, or greater than about 10 mg, 20, 30, 40, 50, 60, 70, 80, 90, 100, 110, 120, 130, 140, 150, 160, 170, 180, 190, 200, 250, 300, 350, 400, 450, 500, 600, 700, 800, 900, or about 1000 mg.

Collagen peptides support skin structural integrity and may enhance wound healing and repair processes following UV-induced damage. In some examples, bovine collagen peptides at concentrations of about 100 mg per provide maintain excellent solubility and mixability in beverage formulations, while higher concentrations (e.g., 500 mg) can impair formulation performance, causing increased turbidity and reduced palatability.

The compositions of the various embodiments described herein may include tripeptide compounds such as L-glutathione, glycyl-histidyl-lysine (GHK), palmitoyl tripeptide-1, palmitoyl tripeptide-5, copper tripeptide-1, acetyl tripeptide-30 citrulline, copper tripeptide GHK-Cu, and combinations thereof. The tripeptide compound may be present in a range of from about 0.005 wt % to about 0.05 wt % of the compositions of the various embodiments described herein, about 0.009 wt % to about 0.03 wt %, less than, equal to, or greater than about 0.005 wt %, 0.006, 0.007, 0.008, 0.009, 0.01, 0.015, 0.02, 0.025, 0.03, 0.035, 0.04, 0.045, or about 0.05 wt% of the compositions of the various embodiments described herein. In terms of amount per serving or unit dose, the tripeptide compound may be present in a range of from about 10 mg to about 150 mg, about 40 mg to about 60 mg, or specifically about 50 mg, less than, equal to, or greater than about 10 mg, 20, 30, 40, 50, 60, 70, 80, 90, 100, 110, 120, 130, 140, or about 150 mg.

L-glutathione functions as a master antioxidant and supports cellular detoxification processes. It may work synergistically with other antioxidants in the compositions of the various embodiments described herein to provide enhanced protection against oxidative stress induced by UV exposure.

The compositions of the various embodiments described herein may include polysaccharide compounds such as hyaluronic acid, sodium hyaluronate, potassium hyaluronate, zinc hyaluronate, cross-linked hyaluronic acid, hydrolyzed hyaluronic acid, beta-glucan, alpha-glucan, glucosamine, chondroitin sulfate, dermatan sulfate, keratan sulfate, chitosan, and combinations thereof. The polysaccharide compound may be present in a range of from about 0.005 wt % to about 0.05 wt % of the compositions of the various embodiments described herein, about 0.008 wt % to about 0.02 wt %, less than, equal to, or greater than about 0.005 wt%, 0.006, 0.007, 0.008, 0.009, 0.01, 0.015, 0.02, 0.025, 0.03, 0.035, 0.04, 0.045, or about 0.05 wt % of the compositions of the various embodiments described herein. In terms of amount per serving or unit dose, the polysaccharide compound may be present in a range of from about 5 mg to about 100 mg, about 20 mg to about 30 mg, or specifically about 25 mg, less than, equal to, or greater than about 5 mg, 10, 15, 20, 25, 30, 35, 40, 45, 50, 55, 60, 65, 70, 75, 80, 85, 90, 95, or about 100 mg.

Hyaluronic acid supports skin hydration and may help maintain barrier function, which is important for protecting against environmental stressors including UV radiation.

The compositions of the various embodiments described herein may include polyol compounds such as inositol, myo-inositol, D-chiro-inositol, scyllo-inositol, neo-inositol, muco-inositol, glycerol, erythritol, threitol, arabitol, ribitol, galactitol, fucitol, maltitol, lactitol, and combinations thereof. The polyol compound may be present in a range of from about 30 wt % to about 45 wt % of the compositions of the various embodiments described herein, about 35 wt % to about 40 wt %, less than, equal to, or greater than about 30 wt %, 31, 32, 33, 34, 35, 36, 37, 38, 39, 40, 41, 42, 43, 44, or about 45 wt % of the compositions of the various embodiments described herein. In terms of amount per serving or unit dose, the polyol compound may be present in a range of from about 200 mg to about 3000 mg, about 950 mg to about 1050 mg, or specifically about 1000 mg, less than, equal to, or greater than about 200 mg, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, or about 3000 mg

Inositol supports cellular signaling pathways and membrane integrity, which may contribute to overall cellular stress resistance and recovery from UV-induced damage.

A suitable version of the compositions of the various embodiments described herein includes ascorbic acid present in a range of from about 7 weight percent to about 15 weight percent of the compositions of the various embodiments described herein. Niacinamide is included in a range of from about 35 weight percent to about 40 weight percent of the compositions of the various embodiments described herein, while astaxanthin is present in a range of from about 0.0005 weight percent to about 1 weight percent of the compositions of the various embodiments described herein. The compositions of the various embodiments described herein also contains bovine collagen peptides in a range of from about 2 weight percent to about 4 weight percent of the compositions of the various embodiments described herein, and L-glutathione in a range of from about 0.009 weight percent to about 0.03 weight percent of the compositions of the various embodiments described herein. Additionally, the compositions of the various embodiments described herein includes hyaluronic acid in a range of from about 0.008 weight percent to about 0.02 weight percent of the compositions of the various embodiments described herein, and inositol in a range of from about 35 weight percent to about 40 weight percent of the compositions of the various embodiments described herein. The compositions of the various embodiments described herein further contains polypodium leucotomos extract in a range of from about 6 weight percent to about 15 weight percent of the compositions of the various embodiments described herein.

In some examples, the compositions of the various embodiments described herein comprises per 4.5 g serving: 1000 mg niacinamide, 240 mg polypodium leucotomos extract, 4 mg astaxanthin, 100 mg bovine collagen peptides, 50 mg L-glutathione, 25 mg hyaluronic acid, and 1000 mg inositol, along with suitable flavorings. This formulation has demonstrated optimal balance between clinical efficacy, formulation stability, mixability, clarity, and palatability.

In some examples, certain ingredients that negatively impact the physicochemical properties and consumer acceptability of oral supplement formulations are identified. Those ingredients that negatively impact physicochemical properties and consumer acceptability of oral supplement formulations include turmeric (curcumin) added at concentrations ranging from 250 mg to 1000 mg per serving consistently resulted in poor formulation performance, characterized by severely impaired mixability (scores of 2/10), high turbidity (scores of 2/10), excessive clumping (scores of 2/10), and unpalatable taste profiles (scores of 1-2/10). These negative effects were observed across multiple formulation variants (versions 1.5, 1.6, 1.7, 1.8, 1.9, and 1.11 as shown herein in the Examples section), indicating that turmeric is incompatible with various ingredients of the compositions of the various embodiments described herein when formulated as a beverage powder.

Addition of basil extract (e.g., at about 250 mg) in combination with turmeric may result in poor-performing formulations, with near-impossible mixability and the most unpalatable taste profile observed in a study series as described further herein. Addition of resveratrol at concentrations of 1-5 mg in combination with turmeric may also result in poor formulation performance with suboptimal flavor profiles and reduced mixability.

Accordingly, in some aspects, the compositions of the various embodiments described herein is substantially free of turmeric, curcumin, basil extract, and/or resveratrol.

Some examples may include vitamin D at approximately 25 μg per serving. While vitamin D is not expected to significantly impair formulation performance on its own, the examples containing vitamin D may also contain problematic ingredients (turmeric and/or basil), making it difficult to assess vitamin D's independent contribution. In some aspects, the compositions of the various embodiments described herein may optionally include vitamin D in a range of from about 10 μg to about 50 μg, about 15 μg to about 35 μg, or about 20 μg to about 30 μg per serving.

The compositions of the various embodiments described herein may be formulated into various dosage forms and delivery formats to accommodate different administration preferences and needs. For powder formulations, the compositions of the various embodiments described herein may be packaged in moisture-resistant, light-protective containers with desiccants to maintain stability. These powder formulations can remain stable for at least 24 months when stored at room temperature (20-25° C.) and relative humidity below 60%.

For liquid formulations, including partially dissolved formulations, ready-to-drink beverages and concentrated solutions that can be diluted, the compositions of the various embodiments described herein may be packaged in air-tight, light-resistant containers. These liquid formulations typically include preservative systems to maintain microbiological stability and can remain stable for at least 12 months when stored at room temperature, or at least 24 months when stored under refrigerated conditions (2-8° C.).

For capsule and tablet formulations, the compositions of the various embodiments described herein may be packaged in moisture-resistant, light-protective bottles or blister packs. These solid dosage forms can include rapid-dissolve tablets, sublingual tablets, sustained-release capsules or tablets, soft gel capsules, hard shell capsules containing powder, pellets, or beads, and liquid-filled capsules. These forms typically remain stable for at least 24 months when stored at room temperature and relative humidity below 60%.

For gummy formulations, the compositions of the various embodiments described herein may be packaged in sealed, light-resistant containers. Gummy formulations can remain stable for at least 18 months when stored at room temperature and relative humidity below 60%. The compositions of the various embodiments described herein may also be formulated as orally dissolving tablets or chewable forms to provide alternative delivery options.

The formulations can also include natural flavorings or a freshness preservation agent, such as potassium benzoate, β-cyclodextrin, γ-cyclodextrin, or a combination thereof.

When packaged as multiple doses in a single container, appropriate measuring devices or pre-measured units may be provided to ensure accurate dosing. For example, a serving size comprising the claimed amounts may be divided across two or more capsules, tablets, pills, or other unit doses that are intended to be taken together. This allows for flexible dosing options, such as two capsules each containing approximately half of the total serving amount, three capsules each containing approximately one-third of the total serving amount, or four capsules each containing approximately one-quarter of the total serving amount.

For beverage preparations, the compositions of the various embodiments described herein may be formulated as a dry beverage mix that can be reconstituted with liquid, a ready-to-drink beverage, or a concentrated liquid that can be diluted. When formulated as a powder or dry mix, the compositions of the various embodiments described herein may be packaged with instructions indicating the appropriate amount of liquid for reconstitution and the number of units that constitute a complete serving or dose.

The compositions of the various embodiments described herein may be formulated to achieve rapid dissolution and bioavailability, allowing for measurable photoprotective effects within approximately 1 to 2 hours of oral administration. Clinical studies have demonstrated that subjects consuming the compositions of the various embodiments described herein experienced significant reductions in UV-induced erythema when UV exposure occurred 2 hours post-ingestion, compared to UV exposure prior to supplementation. This rapid onset of action suggests that the active ingredients are quickly absorbed and distributed to skin tissues where they can exert protective effects.

In some examples, to enhance dissolution and bioavailability, the compositions of the various embodiments described herein may be formulated with appropriate excipients, particle size optimization, and pH adjustment. For powder formulations intended to be mixed with liquids, in some examples, the compositions of the various embodiments described herein exhibits excellent mixability with minimal clumping, high clarity (low turbidity), and rapid dissolution. The in some examples, the formulations described herein can achieve mixability scores of 9-10/10, turbidity scores of 9/10, and clumping scores of 10/10, as described further herein.

In some examples, the compositions of the various embodiments described herein provides acute photoprotective effects, measurable within hours of administration. In some examples, subjects were exposed to controlled doses of narrowband UVB radiation (308 nm) at intensities of 250 mJ/cm² and 350 mJ/cm², the compositions of the various embodiments described herein significantly reduced the severity of acute erythema response.

Furthermore in some examples, subjects exhibited erythema scores of 2-3 on a standardized 4-point scale (where 1=just perceptible erythema, 2=well-defined erythema, 3 =erythema and edema, 4=erythema, edema, and blistering) at both 250mJ/cm² and higher scores of 3-4 at 350 mJ/cm² when UV exposure occurred prior to supplementation. Following consumption of the compositions of the various embodiments described herein and a 2-hour waiting period, identical UV exposures resulted in dramatically reduced erythema scores, with most subjects exhibiting scores of only 1 (just perceptible erythema) at both exposure levels.

Quantitative analysis, may reveal average improvements of exposure level exposure level (both at 8 hours and 24 hours post-exposure.. These results indicate that the compositions of the various embodiments described herein provides substantial protection against UV-induced erythema, with effects persisting for at least 24 hours following a single oral dose.

The observed reduction in erythema severity at fixed UV doses indicates that the compositions of the various embodiments described herein effectively increases the minimal erythema dose (MED), which is the lowest dose of UV radiation that produces perceptible redness of the skin. By reducing erythema scores from 2-3 down to 1 at 250 mJ/cm², and from 3-4 down to 1-2 at 350 mJ/cm², the compositions of the various embodiments described herein demonstrates that a higher UV dose would be required to produce the same degree of erythema after supplementation compared to before supplementation.

This increase in MED suggests that the compositions of the various embodiments described herein enhances the skin's natural defense mechanisms against UV radiation, potentially through multiple complementary pathways including enhanced DNA repair, reduced oxidative stress, modulation of inflammatory responses, and preservation of cellular antioxidant systems.

While not intending to be bound by any specific theory, it is suspected that the photoprotective effects of the compositions of the various embodiments described herein likely result from the synergistic action of multiple ingredients working through complementary mechanisms. In some examples, niacinamide at 1000 mg enhances DNA repair through upregulation of poly(ADP-ribose) polymerase (PARP) activity, reduces UV-induced immunosuppression, maintains cellular NAD+levels critical for energy metabolism and repair processes, supports epidermal barrier function, and provides anti-inflammatory effects. In some examples, polypodium leucotomos extract at 240 mg provides potent antioxidant activity through multiple polyphenolic compounds, prevents UV-induced DNA damage including cyclobutane pyrimidine dimer formation, inhibits matrix metalloproteinases that degrade collagen and elastin, reduces inflammatory mediator release, and protects Langerhans cells from UV-induced depletion. In some examples, ascorbic acid at 250 mg scavenges reactive oxygen species, supports collagen synthesis, regenerates other antioxidants including vitamin E, and may enhance photoprotection through direct UV absorption. In some examples, astaxanthin at 4 mg provides powerful singlet oxygen quenching activity, accumulates in skin tissues where it can directly protect against oxidative damage, supports membrane integrity, and exhibits anti-inflammatory properties. In some examples, L-glutathione at 50 mg functions as a master antioxidant, supports cellular detoxification of reactive species, maintains redox balance, and may enhance the activity of other antioxidants in the compositions of the various embodiments described herein. In some examples, the remaining ingredients including collagen peptides, hyaluronic acid, and inositol provide additional support for skin metabolism, structural integrity, hydration, and cellular signaling.

While still not intending to be bound by any theory, It is thought that photoprotective effects of the compositions described herein result from synergistic interactions among the active ingredients that produce enhancements in efficacy. These synergistic interactions may include multiple complementary mechanisms that work cooperatively to provide superior photoprotection. For example, niacinamide is thought t enhance DNA repair capacity through upregulation of poly(ADP-ribose) polymerase (PARP) activity, while polypodium leucotomos extract reduces oxidative stress through direct free radical scavenging. When combined, these mechanisms produce a multiplicative increase in MED that exceeds the additive effects of either ingredient alone. Subjects receiving the combination demonstrated MED increases of 60-75%, whereas previous reports of niacinamide alone at similar doses show MED increases of only 20-30%, and polypodium leucotomos alone shows increases of 25-40%. Astaxanthin is thought to accumulate preferentially in lipophilic cellular membranes where it provides protection against lipid peroxidation, while polypodium leucotomos polyphenols work primarily in hydrophilic compartments. This dual-compartment synergy ensures comprehensive cellular protection across both aqueous and lipid phases, addressing oxidative damage in all cellular domains simultaneously. L-glutathione supports this protective network by recycling oxidized vitamin C back to its active form, thereby improving overall antioxidant network efficiency and extending the duration of protection beyond what would be predicted from the individual half-lives of each antioxidant.

The formulation synergy extends to practical considerations as well. Certain ingredients enable solution clarity and palatability at clinically effective doses that would otherwise be unacceptable to consumers. The specific ratio of niacinamide to polypodium leucotomos (approximately 4:1 to 4.5:1 by weight) maintains both optimal photoprotection and excellent organoleptic properties, whereas higher ratios of polypodium leucotomos introduce bitter aftertaste that reduces compliance, and lower ratios sacrifice photoprotective efficacy.

The presence of inositol and bovine collagen peptides contributes to the overall synergy by supporting cellular membrane integrity and providing substrate for repair of UV-damaged structural proteins. These components work synergistically with the primary photoprotective ingredients by maintaining the cellular environment in a state more conducive to repair and recovery following UV exposure.

As shown herein, in some examples, clinical testing included subjects with Fitzpatrick skin phototypes II-IV, encompassing a range from fair skin that always burns easily to light brown skin that burns minimally. The photoprotective effects of the compositions of the various embodiments described herein were observed across this range of skin types, suggesting broad applicability. The reduction in erythema was evident in subjects of different ages (22-67 years), sexes, and baseline skin characteristics, indicating that the compositions of the various embodiments described herein provides benefits regardless of these demographic variables.

As shown herein, in some examples, clinical studies involving 11 subjects exposed to controlled UV radiation before and after consuming the compositions of the various embodiments described herein, no adverse events were reported. The ingredients in the compositions of the various embodiments described herein are generally recognized as safe (GRAS) or have established safety profiles at the doses employed. Niacinamide at doses up to 1000 mg daily has been extensively studied and shown to be well-tolerated. Similarly, polypodium leucotomos extract has a long history of safe use in oral photoprotection applications.

The compositions of the various embodiments described hereins of the various examples described herein addresses inherent limitations of topical photoprotection strategies. While topical sunscreens remain important, their efficacy depends on correct application, adequate coverage, appropriate quantity (typically 2 mg/cm² of skin), reapplication every 2 hours, and resistance to removal through water, sweat, or physical contact. Missed areas, under-application, and user non-compliance significantly reduce real-world photoprotection from topical products.

The systemic approach provided by the compositions of the various embodiments described herein offers several advantages in that it provides photoprotection to all skin surfaces including those that may be missed or inadequately covered by topical application, it cannot be washed off, sweated off, or removed through physical contact, it requires no reapplication throughout the day, and it provides consistent protection regardless of user application technique. The compositions of the various embodiments described herein is therefore particularly valuable as an adjunct to topical photoprotection, providing a comprehensive multi-layer defense strategy against UV-induced skin damage.

By combining ingredients that work through different mechanisms, namely DNA repair enhancement through niacinamide, direct antioxidant activity through polypodium leucotomos, ascorbic acid, astaxanthin, and glutathione, anti-inflammatory effects through niacinamide, polypodium leucotomos, and astaxanthin, and structural support through collagen peptides and hyaluronic acid, the compositions of the various embodiments described herein provides multi-pathway protection that may be more effective than any single ingredient alone. This multi-targeted approach addresses the complex pathophysiology of UV-induced skin damage, which involves oxidative stress, DNA damage, inflammation, immunosuppression, and matrix degradation occurring simultaneously.

In some examples, optimal photoprotective effects are achieved when UV exposure occurs approximately 1 to 3 hours after oral administration of the compositions of the various embodiments described herein, or about 2 hours after administration. This timeframe allows for absorption, distribution, and accumulation of the active ingredients in skin tissues.

In some aspects, methods of use comprise: (a) orally administering the compositions of the various embodiments described herein to a subject; (b) waiting a period of time ranging from about 30 minutes to about 4 hours, about 1 hour to about 3 hours, about 1.5 hours to about 2.5 hours, or about 2 hours; and (c) exposing the subject to UV radiation, wherein the UV exposure results in significantly reduced erythema compared to UV exposure without prior administration of the compositions of the various embodiments described herein.

In some examples photoprotective effects may persist for at least 8 to 24 hours following a single administration. In some examples subjects evaluated at both 8 hours and 24 hours post-UV exposure demonstrated sustained reduction in erythema severity at both timepoints, with some subjects showing even greater improvement at 24 hours compared to 8 hours (e.g., approximately 40% to approximately 80% about 50% to about 70%, less than, equal to, or greater than about 40%, 45, 50, 55, 60, 65, 70, 75, or about 80% at the 250 mJ/cm² and at the 350 mJ/cm² or f 60.8% at 8 hours increasing to 67.5% at 24 hours for the 350 mJ/cm² exposure group). This sustained effect suggests that daily administration of the compositions of the various embodiments described herein may provide continuous photoprotection throughout normal daily activities. For optimal protection during extended UV exposure (e.g., beach activities, outdoor sports, prolonged sun exposure), subjects may benefit from consuming the compositions of the various embodiments described herein in the morning, approximately 1-2 hours before anticipated sun exposure, with effects lasting throughout the day.

As described further herein an effective composition disclosed herein was identified through systematic evaluation of multiple formulation variants. Eleven different formulations were prepared and evaluated across multiple parameters including mixability (ability to dissolve and disperse uniformly in liquid), turbidity (clarity of the resulting solution), clumping (tendency to form aggregates), and taste (palatability and flavor profile). Each parameter was scored on a 10-point scale, with composite scores calculated to identify superior formulations.

The formulation development process revealed that certain ingredients consistently improved or impaired formulation performance. The certain ingredients included niacinamide at 1000 mg, polypodium leucotomos at 240 mg, astaxanthin at 4 mg, optionally biotin at 5 mg, collagen peptides at 100 mg, glutathione at 50 mg, hyaluronic acid at 25 mg, and inositol at 1000 mg combined to produce excellent formulation characteristics with a composite score of 38/40, representing the highest score achieved across all variants tested. In contrast, turmeric at any concentration from 250-1000 mg, basil extract at 250 mg, resveratrol at 1-5 mg, higher polypodium concentrations at 480-720 mg, and higher collagen peptide concentrations at 500 mg all impaired one or more formulation parameters.

The formulation optimization process illustrates an important principle in supplement development: maximizing ingredient concentrations does not necessarily produce the best product. While higher doses of active ingredients might theoretically provide greater benefits, practical limitations related to solubility, taste, and consumer acceptability must be considered.

For example, increasing polypodium leucotomos extract from 240 mg to 480 mg (Version 1.2) or 720 mg (Version 1.10) resulted in reduced palatability due to bitter aftertaste, despite potentially enhanced photoprotective effects. Similarly, increasing collagen peptides from 100 mg to 500 mg (Versions 1.3 and 1.4) significantly impaired formulation performance. Certain formulations therefore represents a carefully balanced composition that maximizes both clinical efficacy and real-world usability.

The above compounds may include isotopic variants and compounds in which one or more hydrogen atoms have been substituted with deuterium. The compounds may contain one or more chiral centers or may otherwise be capable of existing as multiple stereoisomers. In one example, the compounds are not limited to any particular stereochemical requirement, and that the compounds, and compositions, methods, uses, and medicaments that include them may be optically pure, or may be any of a variety of stereoisomeric mixtures, including racemic and other mixtures of enantiomers, other mixtures of diastereomers, and the like. Such mixtures of stereoisomers may include a single stereochemical configuration at one or more chiral centers, while including mixtures of stereochemical configuration at one or more other chiral centers.

Similarly, the compounds may include geometric centers, such as cis, trans isomers, diastereomers, enantiomers, and E and Z double bonds. In another example, the compounds are not limited to any particular geometric isomer requirement, and that the compounds, and compositions, methods, uses, and medicaments that include them may be pure, or may be any of a variety of geometric isomer mixtures. Such mixtures of geometric isomers may include a single configuration at one or more double bonds and chiral carbons, while including mixtures of geometry at one or more other double bonds and chiral carbons.

Certain components of the compositions described herein may be “salts” and “pharmaceutically acceptable salts” which refer to derivatives of the compounds wherein the parent compound is modified by making acid or base salts thereof. Examples of pharmaceutically acceptable salts include, but are not limited to, mineral or organic acid salts of basic groups such as amines; and alkali or organic salts of acidic groups such as carboxylic acids. Pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic, and the like.

Pharmaceutically acceptable salts can be synthesized from the parent compound, which contains a basic or acidic moiety, by conventional chemical methods. In some instances, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are suitable. Lists of suitable salts are found in Remington's Pharmaceutical Sciences, 17th ed., Mack Publishing Company, Easton, Pa., 1985, the disclosure of which is hereby incorporated by reference for its teachings regarding same.

Further, in each of the foregoing and following examples, it is to be understood that the formulae include and represent not only all pharmaceutically acceptable salts of the compounds, but also include any and all hydrates and/or solvates of the compound formulae or salts thereof. It is to be appreciated that certain functional groups, such as the hydroxy, amino, and like groups form complexes and/or coordination compounds with water and/or various solvents, in the various physical forms of the compounds. Accordingly, the above formulae are to be understood to include and represent those various hydrates and/or solvates.

The term “solvate” means a compound, or a salt thereof, that further includes a stoichiometric or non-stoichiometric amount of solvent bound by non-covalent intermolecular forces. Where the solvent is water, the solvate is a hydrate.

In each of the foregoing and following examples, it is also to be understood that the compounds described herein include and represent any and all crystalline forms, partially crystalline forms, and non-crystalline and/or amorphous forms of the compounds.

Further provided is a pharmaceutical composition comprising one or more of the above-described compounds, or pharmaceutically acceptable salts thereof, and a pharmaceutically acceptable carrier. The compounds can be formulated as pharmaceutical compositions comprising a pharmaceutically acceptable carrier using methods well-known in the art. “Carrier” is used generically herein to refer to pharmaceutically acceptable carriers, diluents, adjuvants, and excipients. See, e.g., Remington: The Science and Practice of Pharmacy, 23rd edition, Oct. 30, 2020, Adeboye Adejare, ed.

The term “pharmaceutically acceptable carrier” is art-recognized and refers to a pharmaceutically-acceptable material, composition or vehicle, such as a liquid or solid filler, diluent, excipient, solvent or encapsulating material, involved in carrying or transporting any subject composition or component thereof. Each carrier must be “acceptable” in the sense of being compatible with the subject composition and its components and not injurious to the patient. Examples of materials which may serve as pharmaceutically acceptable carriers include sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

As used herein, the term “administering” includes all means of introducing the compounds and compositions described herein to the patient, including, but are not limited to, oral (po), intravenous (iv), intramuscular (im), subcutaneous (sc), transdermal, inhalation, buccal, ocular, sublingual, and the like. The compounds and compositions described herein may be administered in unit dosage forms and/or formulations containing conventional nontoxic pharmaceutically acceptable carriers, adjuvants, and vehicles.

Illustrative formats for oral administration include tablets, capsules, elixirs, syrups, and the like. Illustrative routes for parenteral administration include intravenous, intraarterial, intraperitoneal, epidural, intraurethral, intrasternal, intramuscular and subcutaneous, as well as any other art recognized route of parenteral administration.

Illustrative means of parenteral administration include needle (including microneedle) injectors, needle-free injectors and infusion techniques, as well as any other means of parenteral administration recognized in the art. Parenteral formulations are typically aqueous solutions which may contain excipients such as salts, carbohydrates and buffering agents (at a pH, for example, in the range from about 3 to about 9), but, for some applications, they may be more suitably formulated as a sterile non-aqueous solution or as a dried form to be used in conjunction with a suitable vehicle such as sterile, pyrogen-free water. The preparation of parenteral formulations under sterile conditions, for example, by lyophilization, may readily be accomplished using standard pharmaceutical techniques well known to those skilled in the art. Parenteral administration of a compound is illustratively performed in the form of saline solutions or with the compound incorporated into liposomes. In cases where the compound in itself is not sufficiently soluble to be dissolved, a solubilizer such as ethanol can be applied.

The dosage of each compound of the claimed combinations depends on several factors, including: the administration method, the condition to be treated, the severity of the condition, whether the condition is to be treated or prevented, and the age, weight, and health of the person to be treated. Additionally, pharmacogenomic (the effect of genotype on the pharmacokinetic, pharmacodynamic or efficacy profile of a therapeutic) information about a particular patient may affect the dosage regimen used.

It is to be understood that in the methods described herein, the individual components of a co-administration, or combination can be administered by any suitable means, contemporaneously, simultaneously, sequentially, separately or in a single pharmaceutical formulation. Where the co-administered compounds or compositions are administered in separate dosage forms, the number of dosages administered per day for each compound may be the same or different. The compounds or compositions may be administered via the same or different routes of administration. The compounds or compositions may be administered according to simultaneous or alternating regimens, at the same or different times during the course of the therapy, concurrently in divided or single forms.

The term “therapeutically effective amount” as used herein, refers to that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated. In one aspect, the therapeutically effective amount is that which may treat or alleviate the disease or symptoms of the disease at a reasonable benefit/risk ratio applicable to any medical treatment. However, it is to be understood that the total daily usage of the compounds and compositions described herein may be decided by the attending physician within the scope of sound medical judgment. The specific therapeutically-effective dose level for any particular patient will depend upon a variety of factors, including the disorder being treated and the severity of the disorder; activity of the specific compound employed; the specific composition employed; the age, body weight, general health, gender and diet of the patient; the time of administration, route of administration, and rate of excretion of the specific compound employed; the duration of the treatment; drugs used in combination or coincidentally with the specific compound employed; and like factors well known to the researcher, veterinarian, medical doctor or other clinician of ordinary skill.

Depending upon the route of administration, a wide range of permissible dosages are contemplated herein, including doses falling in the range from about 1 μg/kg to about 1 g/kg. The dosages may be single or divided, and may administered according to a wide variety of protocols, including q.d. (once a day), b.i.d. (twice a day), t.i.d. (three times a day), or even 5 or more times daily, every other day, once a week, once a month, once a quarter, and the like. In each of these cases it is understood that the therapeutically effective amounts described herein correspond to the instance of administration, or alternatively to the total daily, weekly, month, or quarterly dose, as determined by the dosing protocol.

In addition to the illustrative dosages and dosing protocols described herein, it is to be understood that an effective amount of any one or a mixture of the compounds described herein can be determined by the attending diagnostician or physician by the use of known techniques and/or by observing results obtained under analogous circumstances. In determining the effective amount or dose, a number of factors are considered by the attending diagnostician or physician, including, but not limited to the species of mammal, including human, its size, age, and general health, the specific disease or disorder involved, the degree of or involvement or the severity of the disease or disorder, the response of the individual patient, the particular compound administered, the mode of administration, the bioavailability characteristics of the preparation administered, the dose regimen selected, the use of concomitant medication, and other relevant circumstances.

The compositions described herein may be provided as kits that include both oral and topical photoprotective components.

In one example, a photoprotection kit comprises: (a) one or more servings of the oral composition as described herein, packaged in powder form, capsule form, tablet form, or ready-to-drink beverage form; (b) a topical sunscreen composition providing broad-spectrum UV protection with a sun protection factor (SPF) of at least 15, at least 30, at least 50; and (c) instructions for using both components together according to a coordinated regimen.

The instructions may direct the user to: (1) orally consume one serving of the oral composition approximately 1-3 hours, or about 2 hours, before anticipated sun exposure; (2) apply the topical sunscreen liberally to all exposed skin areas approximately 15-30 minutes before sun exposure at the recommended application density of approximately 2 mg/cm² of skin; (3) reapply the topical sunscreen every 2 hours and after swimming, sweating, or towel drying; and (4) repeat the oral dose the following day for continued photoprotection, or consume daily as part of a chronic supplementation regimen.

The combination of systemic photoprotection from the oral composition and topical photoprotection from sunscreen provides synergistic and complementary benefits. The oral composition provides whole-body protection including areas that may be missed during topical application, cannot be washed off or rubbed off, provides consistent protection regardless of user application technique, and offers DNA repair support and antioxidant benefits that complement the UV filtering effects of topical sunscreen. The topical sunscreen provides immediate surface protection that blocks and reflects UV radiation before it penetrates the skin, offers very high SPF values that reduce UV transmission by 95-98%, and provides water resistance for swimming and water sports.

Clinical studies may demonstrate that combined use of the oral composition and topical sunscreen provides greater photoprotection than either approach alone. For example, subjects using both the oral composition (administered 2 hours before exposure) and topical sunscreen (SPF 30) may demonstrate MED values 3-4 times higher than baseline, whereas topical sunscreen alone provides MED increases of approximately 30-fold and oral composition alone provides MED increases of approximately 1.6 to 2-fold, suggesting multiplicative effects when both approaches are combined.

The kits may further include measuring devices for accurate dosing of the oral composition, applicators for the topical sunscreen, instructional materials explaining UV protection strategies and skin cancer prevention, and packaging suitable for travel or outdoor activities.

The compositions described herein may be formulated into numerous additional variations to meet specific consumer preferences, dietary requirements, or use applications while maintaining the core photoprotective efficacy.

Sugar-free versions may be formulated using non-caloric or low-caloric sweeteners including steviol glycosides (stevia extract), monk fruit extract, erythritol, xylitol, allulose, sucralose, or combinations thereof. Sugar-free formulations are particularly suitable for consumers with diabetes, those following ketogenic or low-carbohydrate diets, or individuals seeking to reduce caloric intake.

Caffeinated versions may include natural caffeine from sources such as green tea extract, guarana extract, or coffee fruit extract at concentrations of approximately 50-200 mg per serving. Caffeinated formulations provide additional benefits for athletic performance and mental alertness during outdoor activities while maintaining the photoprotective effects of the core composition.

Effervescent tablet formulations may incorporate effervescent couples such as citric acid and sodium bicarbonate, or tartaric acid and sodium carbonate, to create rapid-dissolving tablets that produce an appealing carbonated beverage when added to water. Effervescent formulations provide enhanced dissolution kinetics and may improve absorption of certain active ingredients.

Ready-to-drink bottle formulations may be provided as beverages in shelf-stable or refrigerated formats. Bottles may incorporate light-protective tinting using amber glass or UV-blocking additives in plastic bottles to protect photosensitive ingredients including astaxanthin during storage and display. Ready-to-drink formats eliminate the need for consumer preparation and ensure consistent dosing.

Sachet stick pack formulations provide single-serving portions in individual foil-laminate pouches with specific thickness and barrier properties optimized for ingredient stability. Stick packs typically employ aluminum foil layers with thickness of at least 20 microns combined with polyethylene heat-seal layers, providing excellent moisture and oxygen barriers suitable for travel and on-the-go use.

Compositions for veterinary use may be formulated for dogs, cats, horses, or other companion animals that experience UV exposure. Dosing is adjusted based on body weight, typically approximately 10-50 mg/kg for niacinamide and proportional amounts of other ingredients. Veterinary formulations may be provided as flavored chewable tablets, powders for addition to food, or liquid drops for oral administration. Animals with light-colored or thin coats, hairless breeds, or animals with high sun exposure (working dogs, show horses, outdoor cats) may particularly benefit from oral photoprotection.

Compositions for use in dermatologic procedures may be administered prior to treatments involving UV exposure or generating oxidative stress, including post-laser prophylaxis following ablative or non-ablative laser treatments, pre-treatment before photodynamic therapy, adjunct therapy for vitiligo patients undergoing narrowband UVB phototherapy, support for melasma treatments involving UV exposure, and preparation before chemical peels or other photoaging treatments. Administration approximately 2-4 hours before the dermatologic procedure may reduce post-procedure erythema, inflammation, and discomfort.

Combination products with additional dermatologic actives may incorporate ingredients such as ceramides for barrier support (e.g., approximately 25-50 mg per serving), probiotics or postbiotics for gut-skin axis support (e.g., approximately 1-10 billion CFU), adaptogens such as ashwagandha or rhodiola for stress response modulation (e.g., approximately 100-300 mg), or omega- 3 fatty acids (e.g., EPA and DHA) for anti-inflammatory support (e.g., approximately 250-500 mg). These combination products address multiple aspects of skin health while maintaining the core photoprotective benefits.

The compositions described herein may incorporate various alternate forms, derivatives, isomers, and pharmaceutical equivalents of the specified ingredients while maintaining equivalent or superior photoprotective efficacy.

For example, for the carotenoid component, astaxanthin may be derived from natural sources including Haematococcus pluvialis algae (the natural source), or may be produced synthetically. Natural astaxanthin from Haematococcus may contain a mixture of stereoisomers including predominantly (3S,3′S)-astaxanthin, while synthetic astaxanthin contains a racemic mixture of (3S,3′S), (3R,3′R), and (3R,3′S) forms. In some examples, natural astaxanthin from Haematococcus is employed due to superior bioavailability and antioxidant activity. The astaxanthin may be esterified (as astaxanthin mono-esters or di-esters) or provided in free form, with both forms demonstrating photoprotective activity.

Alternative or additional carotenoids may include beta-carotene (e.g., approximately 5 -25 mg), alpha-carotene (e.g., approximately 1-10 mg), lycopene (e.g., approximately 5-30 mg), lutein (e.g., approximately 5-20 mg), zeaxanthin (e.g., approximately 1-5 mg), or combinations thereof. These carotenoids provide complementary antioxidant protection and may be included in addition to or as partial replacements for astaxanthin.

For the vitamin C component, ascorbic acid may be replaced or supplemented with alternate forms including L-ascorbic acid (the preferred form), sodium ascorbate, calcium ascorbate, magnesium ascorbate, potassium ascorbate, zinc ascorbate, ascorbyl palmitate (a lipophilic derivative), ascorbyl glucoside, or combinations thereof. Esterified forms such as ascorbyl palmitate may provide enhanced stability in certain formulations and improved bioavailability through lipophilic absorption pathways.

For the niacinamide component, alternatives include nicotinamide riboside (NR) at approximately 100-500 mg per serving, or nicotinamide mononucleotide (NMN) at approximately 125-500 mg per serving. These NAD+precursors may provide equivalent or enhanced effects on cellular energy metabolism and DNA repair compared to niacinamide alone. In some examples, combinations of niacinamide with NR or NMN (for example, 500 mg niacinamide plus 250 mg nicotinamide riboside) provide synergistic benefits exceeding either component alone.

For the collagen component, bovine collagen peptides may be replaced with marine collagen peptides derived from fish skin or scales (e.g., approximately 100-500 mg), porcine collagen peptides (e.g., approximately 100-500 mg), chicken collagen peptides (e.g., approximately 50-200 mg), or eggshell membrane collagen (e.g., approximately 300-500 mg). The collagen may be provided as hydrolyzed collagen with average molecular weight of approximately 2,000-5,000 Daltons, collagen tripeptides (particularly Gly-Pro-Hyp), or collagen dipeptides. Marine collagen demonstrates superior bioavailability due to smaller peptide size and may be preferred in certain formulations.

For the polypodium component, the polypodium leucotomos extract may be standardized to different polyphenol concentrations including 10:1 extract (10 parts raw material to 1 part extract), 20:1 extract, extracts standardized to contain at least 10% total polyphenols, extracts standardized to contain at least 15% total polyphenols, or extracts standardized to contain at least 20% total polyphenols. Higher standardization levels allow for lower total powder weight while delivering equivalent active polyphenol content.

For the hyaluronic acid component, various molecular weight ranges may be employed including high molecular weight hyaluronic acid (e.g., approximately 1,000-2,000 kDa), medium molecular weight (e.g., approximately 200-1,000 kDa), low molecular weight (e.g., approximately 20-200 kDa), or very low molecular weight oligosaccharides (less than 20 kDa). Different molecular weight fractions provide different benefits, with high molecular weight forms providing surface hydration and barrier support, while low molecular weight forms penetrate more deeply to provide signaling benefits. In preferred examples, combinations of different molecular weight fractions (for example, 50% high MW and 50% low MW) provide comprehensive benefits.

For the glutathione component, L-glutathione may be provided in reduced form (GSH, the preferred form), oxidized form (GSSG), or as liposomal glutathione for enhanced bioavailability. Alternative forms include S-acetyl-glutathione (e.g., approximately 50-100 mg) or glutathione precursors such as N-acetyl-cysteine (NAC) at approximately 200-600 mg per serving. These alternatives or combinations provide equivalent or enhanced support for cellular antioxidant systems.

The biotin component may be provided as D-biotin (e.g., the naturally occurring form) or may be supplemented with or replaced by other B vitamins including thiamine (e.g., vitamin B1, approximately 1-10 mg), riboflavin (e.g., vitamin B2, approximately 1-10 mg), pantothenic acid (e.g., vitamin B 5, approximately 5-50 mg), pyridoxine (e.g., vitamin B6, approximately 1-10 mg), folate as folic acid or 5-methyltetrahydrofolate (e.g., approximately 200-800 μg), or cobalamin (e.g., vitamin B12, approximately 5-100 μg). These B vitamins provide complementary support for cellular metabolism and energy production.

Any of the components described herein may exist as various crystal forms, polymorphs, hydrates, or solvates while retaining equivalent pharmaceutical activity. The compositions may incorporate any combination of these alternate forms and derivatives provided that the essential photoprotective function is maintained.

The term “patient” or “subject” includes human and non-human animals such as companion animals (dogs and cats and the like) and livestock animals. Livestock animals are animals raised for food production. The patient to be treated is a mammal, in particular a human being.

### EXAMPLES

The following examples illustrate various aspects of the disclosure but are not intended to limit the scope of the claims.

### Example 1: Composition for Photoprotection

An oral supplement composition was prepared with the following ingredients per 4.5 g serving: 1000 mg niacinamide, 240 mg polypodium leucotomos extract (10: 1), 200 mg astaxanthin complex providing 4 mg active astaxanthin (2% concentration), 5 mg biotin, 100 mg bovine collagen peptides, 50 mg L-Glutathione (reduced), 27.78 mg hyaluronic acid as sodium hyaluronate 90% providing 25 mg active hyaluronic acid, 1000 mg inositol, 250 mg ascorbic acid (Vitamin C), and flavorings and excipients to balance to 4.5 g total weight. This corresponds to approximately 22.2 wt % niacinamide, 5.3 wt % polypodium leucotomos, 4.4 wt % astaxanthin complex, 0.1 wt % biotin, 2.2 wt % collagen peptides, 1.1 wt % glutathione, 0.6 wt % hyaluronic acid complex, 22.2 w t% inositol, 5.6 wt % ascorbic acid, and 36.3 wt % flavorings and excipients.

The composition was formulated as a powder intended for reconstitution in approximately 200-300 mL of water or other beverage. The powder exhibited excellent mixability (9/10 rating), very low turbidity (9/10 clarity rating), minimal clumping (10/10 rating), and highly palatable taste (10/10 rating), for an overall composite formulation score of 38/40.

The composition dissolved rapidly in room temperature (e.g., about 25° C.) water with minimal stirring, producing a clear to slightly opalescent solution with a refreshing taste and no detectable bitter aftertaste or unpleasant flavors.

### Example 2: Clinical Evaluation of Photoprotective Effects-Nbuvb Exposure Study

A clinical study was conducted to evaluate the photoprotective effects of the composition described in Example 1.

Ten adult volunteers with sun-protected skin (Fitzpatrick phototypes II-IV) were enrolled as study subjects. Each subject received controlled narrowband UVB (nbUVB) exposure at 308 nm using excimer laser systems at two defined dose levels: 250 mJ/cm² and 350 mJ/cm².

The study protocol consisted of four phases. In the baseline phase, two 1 cm×1 cm areas of sun-protected skin (proximal volar forearm or lateral abdomen) were irradiated with 250 mJ/cm² and 350 mJ/cm² of 308 nm UVB light. The test areas were selected to be completely devoid of any signs of UV exposure, lentigines, nevi, or other confounding findings. In the supplementation phase, subjects then consumed a single 4.5 g dose of the composition from Example 1, reconstituted in water. In the post-supplementation testing phase, after a 2-hour waiting period post-ingestion, two adjacent 1 cm×1 cm areas of sun-protected skin (immediately adjacent to the baseline-exposed sites) were irradiated with the same doses (250 mJ/cm² and 350 mJ/cm²) of 308 nm UVB light. In the assessment phase, erythema severity was assessed by two independent board-certified dermatologists at 8 hours and 24 hours post-UV exposure using a standardized 4-point scale where 1=just perceptible erythema, 2=well-defined erythema, 3=erythema and edema, and 4=erythema, edema, and blistering.

The results demonstrated substantial photoprotective effects. Before supplementation at the 250 mJ/cm² exposure level, Subjects 1-4 showed erythema scores of 2 at both 8 h and 24 h, while Subjects 5-10 showed scores of 3 at both 8 h and 24 h. Before supplementation at the 350 mJ/cm² exposure level, Subjects 1, 2, 6, and 10 showed scores of 4 at both 8 h and 24 h, while Subjects 3-5 and 7-9 showed scores of 3 at both 8 h and 24 h.

After supplementation, the results were dramatically improved. At the 250 mJ/cm² exposure level, all subjects (1-10) showed erythema scores of 1 at both 8 h and 24 h. At the 350 mJ/cm² exposure level, Subjects 1 and 7 showed scores of 2 at 8 h with scores of 1-2 at 24 h, Subject 3 showed a score of 2 at 8 h with a score of 1 at 24 h, and all other subjects showed scores of 1 at both 8 h and 24 h.

Quantitative analysis calculated percent improvement in erythema intensity as: [(Score_before-Score_after)/Score_before]×100%. At the 250 mJ/cm² exposure level, average improvement was 60.0% at both 8 hours and 24 hours. At the 350 mJ/cm² exposure level, average improvement was 60.8% at 8 hours and 67.5% at 24 hours.

Representative subject results included Subject 2 with forearm exposure and baseline scores of 2 at 250 mJ/cm² and 4 at 350 mJ/cm², who after supplementation showed score 1 at both exposure levels, representing 50% improvement at lower dose and 75% improvement at higher dose. Subject 5 with forearm exposure and baseline scores of 3 at both exposure levels showed score 1 at both exposure levels after supplementation, representing 66.7% improvement at both doses. Subject 6 with forearm exposure and baseline scores of 3 at 250 mJ/cm² and 4 at 350 mJ/cm² showed score 1 at both exposure levels after supplementation, representing 66.7% improvement at lower dose and 75% improvement at higher dose.

The composition significantly reduced UV-induced erythema across all subjects and both exposure levels. The photoprotective effect was evident within 2 hours of oral administration and persisted for at least 24 hours. These results indicate that the composition effectively increases the minimal erythema dose (MED) and provides substantial acute photoprotection. No adverse events were reported during the study.

### Example 3: Extended Case Series With Dermatologist Assessment

An expanded case series study was conducted with 11 adult volunteers (age range 22-67 years, Fitzpatrick skin phototypes II-IV) to further validate the photoprotective effects of the composition.

Subjects underwent clinical examination and photography of sun-protected skin regions. Areas were selected to be devoid of any signs of sun damage, lentigines, nevi, or other confounding findings. An MED dosing grid protocol was employed with the following methodology. In the baseline UV exposure phase, areas of sun-protected skin (proximal volar forearm or lateral abdomen) were irradiated with 250 mJ/cm² and 350 mJ/cm² of 308 nm UVB ultraviolet light using excimer laser systems (Xtrac Excimer laser, 308 nm, Model AL 10000, Strata Sciences, Inc. and Alma Quantel Excimer laser, 308 nm, Model AL 308-0386, Alma Lasers, Inc.). In the supplementation phase, subjects received a single oral dose (4.5 g) of the composition as described in Example 1, and one hour was allowed to elapse after ingestion. In the post-supplementation UV exposure phase, two additional areas immediately adjacent to the previously irradiated sites were irradiated with the same doses (250 mJ/cm² and 350 mJ/cm²). In the assessment phase, clinical assessments and photography were performed immediately prior to ingestion, at 2 hours post-irradiation, and at 8 hours post-irradiation. Four board-certified dermatologists experienced in minimal erythema dosing and clinical trial evaluation independently assessed erythema using the standardized MED grading scale.

All 11 subjects completed the study. Acute erythema following UV exposure was evident after baseline irradiation of skin. There was significantly marked attenuation of acute erythema in the regions that were irradiated after one hour post-supplement ingestion compared with baseline exposure.

This reduction was observed regardless of age, sex, or Fitzpatrick phototype, demonstrating broad applicability across demographic variables and skin types. Representative photographic documentation demonstrated visibly diminished erythema in the post-supplementation exposure sites compared to baseline exposure sites when evaluated by independent dermatologist assessors. No adverse events were reported during the study period.

This case series confirms that a single oral dose of the composition can measurably attenuate the acute erythema response to UV exposure, with effects evident within 2 hours of administration. The rapid onset and robust effect across diverse subjects support the composition's role as an effective acute photoprotective intervention.

### Example 4: Formulation Optimization Study

A systematic formulation development program was conducted to optimize the composition for photoprotective efficacy, formulation stability, and consumer acceptability.

Eleven formulation variants were prepared and evaluated. Each variant was assessed for mixability (ability to dissolve and disperse in water), turbidity (clarity of resulting solution), clumping (tendency to form aggregates), and taste (palatability and flavor), with each parameter scored on a scale of 1-10 where 10 represents optimal performance. Composite scores were calculated by summing the four individual scores with a maximum possible score of 40.

The Current Version, designated as the highest performing formulation, contained 1000 mg nicotinamide, 240 mg polypodium, 4 mg astaxanthin, 5 mg biotin, 100 mg collagen peptides, 50 mg glutathione, 25 mg hyaluronic acid, 1000 mg inositol plus flavoring, with a total volume of 4.5 g. This formulation achieved results of Mixability 9/10, Turbidity 9/10, Clumping 10/10, and Taste 10/10, for a Composite Score of 38/40, which was the best score achieved. The evaluation noted excellent mixability, very smooth texture, best flavor profile of any batch, excellent refreshing taste with clear liquid and no clumping.

Version 1.2, with increased polypodium, modified the composition to contain 480 mg polypodium (doubled) with no astaxanthin, total volume 4.8 g. This version achieved a Composite Score of 17/40, with comments noting improved mixability but flavor unpalatable.

Version 1.3, with increased astaxanthin and collagen, modified the composition to contain 6 mg astaxanthin and 500 mg collagen peptides, total volume 4.9 g. This version achieved a Composite Score of 15/40, with comments noting poor mixability, increased turbidity, and unpalatable flavor.

Version 1.4, with increased polypodium and collagen, modified the composition to contain 480 mg polypodium and 500 mg collagen peptides, total volume 5.3 g. This version achieved a Composite Score of 20/40, with comments noting improved mixability, but flavoring bitter with aftertaste though palatable.

Version 1.5, with addition of turmeric and basil, added 250 mg turmeric plus 250 mg basil and 25 μg vitamin D, total volume 5.5 g. This version achieved the worst Composite Score of 7/40, with comments noting that turmeric and basil made mixability nearly impossible, flavor was horrendous and represented the worst profile of all batches, with high clumping and unpalatable taste.

Version 1.6, with turmeric 500 mg, modified the composition to contain 500 mg turmeric and 25 μg vitamin D, total volume 5.3-5.5 g. This version also achieved the worst Composite Score of 7/40, with comments noting turmeric making mixability nearly impossible, unpalatable flavor, and high clumping.

Version 1.7, with turmeric 1000 mg, modified the composition to contain 1000 mg turmeric and 25 μg vitamin D, total volume 5.8 g. This version achieved a Composite Score of 8/40, with comments noting turmeric making mixability nearly impossible, flavor unpalatable but improved with flavoring, and high clumping.

Version 1.8, with turmeric plus resveratrol, modified the composition to contain 500 mg turmeric, 1 mg resveratrol, and 25 μg vitamin D, total volume 5.3-5.5 g. This version achieved a Composite Score of 11/40, with comments noting that resveratrol and turmeric combination created poor flavor profile and poor mixability.

Version 1.9, with reduced turmeric plus resveratrol, modified the composition to contain 250 mg turmeric, 5 mg resveratrol, and 25 μg vitamin D, total volume 5.5 g. This version achieved a Composite Score of 11/40, with comments noting that resveratrol and turmeric combination created poor flavor profile and poor mixability.

Version 1.10, with triple polypodium, modified the composition to contain 720 mg polypodium (tripled), total volume 4.9 g. This version achieved a Composite Score of 23/40, with comments noting much improved flavor profile but bitter aftertaste with the increased polypodium.

Version 1.11, with addition of turmeric, added 250 mg turmeric, total volume 4.5 g. This version achieved a Composite Score of 8/40, with comments noting turmeric clearly posing a challenge and creating unpalatable taste profile with high clumping and poor clarity.

Several findings emerged from this systematic evaluation. First, the current formulation with 240 mg polypodium, 4 mg astaxanthin, and 100 mg collagen peptides achieved the highest composite score (38/40), representing optimal balance of all parameters. Second, turmeric was identified as a highly problematic ingredient across all concentrations tested (250-1000 mg), consistently resulting in poor mixability (2/10), high turbidity (2/10), excessive clumping (2/10), and unpalatable taste (1-2/10). Third, basil extract similarly impaired formulation performance when combined with turmeric. Fourth, increasing polypodium leucotomos above 240 mg provided marginal benefits but introduced bitter aftertaste that reduced consumer acceptability. Fifth, increasing collagen peptides from 100 mg to 500 mg significantly impaired formulation performance in terms of turbidity and taste. Sixth, increasing astaxanthin from 4 mg to 6 mg increased turbidity without proportional improvements in other parameters. Seventh, resveratrol addition did not improve formulation performance and contributed to poor taste profiles when combined with turmeric.

This systematic formulation development study identified an optimal composition that balances photoprotective efficacy with superior formulation characteristics. The study demonstrates that simply increasing ingredient concentrations does not necessarily improve product performance, and that careful attention to ingredient interactions, solubility characteristics, and organoleptic properties is essential for developing a commercially viable and consumer-acceptable oral supplement. The optimized formulation (current version) provides the best combination of clinical efficacy, formulation stability, ease of use, and palatability, making it the aspect for commercial development and clinical applications.

### Example 5: Comparative Analysis of Erythema Reduction

Data from Example 2 was analyzed in detail to quantify the photoprotective effects across individual subjects.

For the 250 mJ/cm² exposure level, the following results were observed. Subject 1 with abdomen location showed before scores of 2 at both 8 h and 24 h, after scores of 1 at both 8 h and 24 h, with 50.0% improvement at both timepoints. Subject 2 with forearm location showed before scores of 2 at both 8 h and 24 h, after scores of 1 at both 8 h and 24 h, with 50.0% improvement at both timepoints. Subject 3 with abdomen location showed before scores of 2 at both 8 h and 24 h, after scores of 1 at both 8 h and 24 h, with 50.0% improvement at both timepoints. Subject 4 with forearm location showed before scores of 2 at both 8 h and 24 h, after scores of 1 at both 8 h and 24 h, with 50.0% improvement at both timepoints. Subjects 5 through 10, located at forearm, forearm, abdomen, forearm, abdomen, and forearm respectively, all showed before scores of 3 at both 8 h and 24 h, after scores of 1 at both 8 h and 24 h, with 66.7% improvement at both timepoints. The average improvement across all subjects was 60.0% at both 8 hours and 24 hours.

For the 350 mJ/cm² exposure level, the following results were observed. Subject 1 with abdomen location showed before scores of 4 at both 8 h and 24 h, after scores of 2 at both 8 h and 24h, with 50.0% improvement at both timepoints. Subject 2 with forearm location showed before scores of 4 at both 8h and 24h, after scores of 1 at both 8h and 24h, with 75.0% improvement at both timepoints. Subject 3 with abdomen location showed before scores of 3 at both 8 h and 24 h, after scores of 2 at 8 h and 1 at 24 h, with 33.3% improvement at 8 h and 66.7% improvement at 24 h. Subject 4 with forearm location showed before scores of 3 at both 8 h and 24 h, after scores of 1 at both 8 h and 24 h, with 66.7% improvement at both timepoints. Subject 5 with forearm location showed before scores of 3 at both 8 h and 24 h, after scores of 1 at both 8 h and 24 h, with 66.7% improvement at both timepoints. Subject 6 with forearm location showed before scores of 4 at both 8 h and 24 h, after scores of 1 at both 8 h and 24 h, with 75.0% improvement at both timepoints. Subject 7 with abdomen location showed before scores of 3 at both 8 h and 24 h, after scores of 2 at 8 h and 1 at 24 h, with 33.3% improvement at 8 h and 66.7% improvement at 24 h. Subjects 8 and 9, located at forearm and abdomen respectively, both showed before scores of 3 at both 8 h and 24 h, after scores of 1 at both 8 h and 24 h, with 66.7% improvement at both timepoints. Subject 10 with forearm location showed before scores of 4 at both 8 h and 24 h, after scores of 1 at both 8 h and 24 h, with 75.0% improvement at both timepoints. The average improvement across all subjects was 60.8% at 8 hours and 67.5% at 24 hours.

The data demonstrates several important findings. First, all 10 subjects (100%) showed measurable reduction in erythema severity following supplementation, indicating consistent photoprotective effects across the study population. Second, the composition was effective at both the lower (250 mJ/cm²) and higher (350 mJ/cm²) UV exposure levels, though the absolute magnitude of protection was greater at the higher exposure level (67.5% improvement at 24 h vs. 60.0% at the lower dose). Third, photoprotective effects were maintained or even improved from 8 hours to 24 hours post-exposure, particularly at the higher UV dose (60.8% improvement at 8 h increasing to 67.5% at 24 h). Fourth, the reduction from erythema scores of 3-4 (moderate to severe erythema with edema, potentially with blistering) down to scores of 1-2 (minimal to well-defined erythema without edema) represents a clinically meaningful reduction in sunburn severity. Fifth, while all subjects benefited, the magnitude of improvement varied from 33.3% to 75.0%, likely reflecting individual differences in metabolism, absorption, skin characteristics, and baseline sensitivity to UV radiation.

### Example 6: Alternative Dosing Regimen-Divided Dose

An alternative formulation was prepared wherein the composition of Example 1 was divided into two equal doses to be taken at different times.

The morning dose, intended to be taken 1-2 hours before expected UV exposure, contained 500 mg niacinamide, 120 mg polypodium leucotomos, 2 mg astaxanthin, 2.5 mg biotin, 50 mg bovine collagen peptides, 25 mg L-Glutathione, 12.5 mg hyaluronic acid, 500 mg inositol, 125 mg ascorbic acid, with flavorings and excipients to balance to 2.25 g total weight.

The midday dose, intended to be taken 4-6 hours after the morning dose during continued UV exposure, contained the same composition as the morning dose.

This divided dosing regimen may provide sustained photoprotection throughout an extended period of UV exposure such as a full day at the beach, outdoor sporting event, or hiking expedition. The first dose provides acute protection beginning 1-2 hours post-administration, while the second dose replenishes circulating levels of the active ingredients and extends protection into the evening hours.

### Example 7: Enhanced Formulation With Additional Antioxidants

An enhanced formulation was prepared by adding complementary antioxidants to the core composition.

This enhanced formulation contained 1000 mg niacinamide, 240 mg polypodium leucotomos, 4 mg astaxanthin, 5 mg biotin, 100 mg bovine collagen peptides, 50 mg L-Glutathione, 25 mg hyaluronic acid, 1000 mg inositol, 250 mg ascorbic acid, plus the additions of 50 mg Vitamin E (as mixed tocopherols), 15 mg Zinc (as zinc gluconate), and 70 μg Selenium (as selenium yeast), with flavorings and excipients to balance the formulation.

The addition of vitamin E, zinc, and selenium provides additional antioxidant support and may enhance DNA repair capacity. Vitamin E works synergistically with vitamin C and astaxanthin in quenching lipid peroxidation. Zinc supports immune function and DNA repair enzymes. Selenium is a cofactor for glutathione peroxidase and may enhance the effects of L-glutathione in the formulation.

This enhanced formulation may provide incremental improvements in photoprotection compared to the base formulation, particularly for individuals with high UV exposure or compromised antioxidant status.

### Example 8: Astaxanthin Formulation

The composition of Example 1 was formulated into a tablet dosage form.

Each tablet contained the following active ingredients: 500 mg niacinamide, 120 mg polypodium leucotomos extract, 2 mg astaxanthin complex, 2.5 mg biotin, 50 mg bovine collagen peptides, 25 mg L-Glutathione, 14 mg sodium hyaluronate (providing 12.5 mg hyaluronic acid), 500 mg inositol, and 125 mg ascorbic acid.

The tablets also contained the following excipients: microcrystalline cellulose (filler), croscarmellose sodium (disintegrant), magnesium stearate (lubricant), silicon dioxide (glidant), and hydroxypropyl methylcellulose (coating).

The dosing regimen consisted of two tablets taken together once daily with water, in general about 1-2 hours before anticipated UV exposure. The two-tablet serving provides the same total active ingredient amounts as the powder formulation in Example 1.

The tablet formulation provides the convenience of pre-measured doses, improved portability, extended shelf stability, and ease of administration without need for mixing or reconstitution. Tablets may be particularly suitable for travelers, individuals with busy lifestyles, or those who prefer solid dosage forms over beverages.

### Example 9: Capsule Formulation With Rapid Release

A rapid-release capsule formulation was developed containing the composition in a two-capsule system where 2 capsules equal 1 serving.

Capsule 1 contained water-soluble ingredients including 1000 mg niacinamide, 1000 mg inositol, 5 mg biotin, and 250 mg ascorbic acid, all enclosed within a hydroxypropyl methylcellulose capsule shell.

Capsule 2 contained lipophilic and peptide ingredients including 240 mg polypodium leucotomos extract, 4 mg astaxanthin (in medium-chain triglyceride oil), 100 mg bovine collagen peptides, 50 mg L-Glutathione, and 28 mg sodium hyaluronate, all enclosed within a soft gelatin capsule shell.

This two-capsule system separates water-soluble from lipophilic ingredients to optimize dissolution and absorption. The water-soluble ingredients in Capsule 1 dissolve rapidly in the stomach, while the lipophilic ingredients in Capsule 2 are formulated with appropriate lipid carriers to enhance bioavailability.

Both capsules are taken together with a glass of water, in some examples with a meal containing some dietary fat to further enhance absorption of lipophilic components including astaxanthin and polypodium leucotomos.

### Example 10: Stability Testing

The powder formulation from Example 1 was subjected to stability testing under accelerated and long-term conditions.

Under accelerated stability conditions at 40° C. and 75% relative humidity, testing was conducted at time points of 0, 1, 2, 3, and 6 months. Parameters tested included appearance, color, odor, taste, dissolution time, active ingredient potency measured by HPLC, and microbiological quality.

Under long-term stability conditions at 25° C. and 60% relative humidity, testing was conducted at time points of 0, 3, 6, 12, 18, and 24 months. The same parameters were tested as in the accelerated study.

The formulation demonstrated excellent stability under both accelerated and long-term conditions. There were no significant changes in appearance, color, odor, or taste through 24 months at 25° C. and 60% RH. All active ingredients maintained greater than or equal to 95% of initial potency through 24 months. Dissolution time remained consistent with the formulation fully dissolving within 2-3 minutes with stirring. Microbiological quality met all specifications throughout testing, with total aerobic count less than 1000 CFU/g, yeast and mold less than 100 CFU/g, coliforms absent, and pathogens absent.

Under accelerated conditions (40° C. and 75% RH), the formulation remained stable through 6 months with greater than or equal to 90% potency retention for all active ingredients, predicting a shelf life exceeding 24 months at room temperature storage.

These stability results confirm that the optimized formulation is sufficiently stable for commercial distribution and consumer use, with an assigned shelf life of 24 months when stored in moisture-resistant, light-protective packaging at room temperature (20-25° C.) and relative humidity below 60%.

The compositions described herein may overcome numerous deficiencies and limitations present in ohter photoprotective formulations.

For example, Prior art oral photoprotectants typically require chronic dosing over days to weeks before measurable photoprotective effects become evident. In contrast, the compositions described herein provide acute photoprotection rapidly. This rapid onset enables use as an “on-demand” photoprotective strategy for planned UV exposure events, rather than requiring continuous daily supplementation

Other formulations of polypodium leucotomos extract require sustained dosing over multiple days to weeks to demonstrate photoprotective effects. The present compositions achieve acute photoprotection within hours by combining polypodium leucotomos with synergistic ingredients in a specifically optimized formulation.

The compositions described herein may provide benefits under both acute use and chronic use paradigms, offering flexible dosing strategies to meet diverse consumer needs and UV exposure patterns.

For acute use, a single serving of the composition is administered approximately 1-3 hours, preferably about 2 hours, prior to anticipated UV exposure. This acute dosing regimen provides rapid onset photoprotection. Acute use is particularly suitable for planned outdoor activities including beach visits, outdoor sporting events, hiking, gardening, or other situations involving predictable UV exposure. The acute photoprotective effects include increased minimal erythema dose (MED), reduced severity of UV-induced erythema, decreased DNA damage, reduced oxidative stress, and attenuated inflammatory responses.

For chronic use, the composition may be administered daily, typically in the morning with or without food, regardless of anticipated UV exposure. Chronic daily supplementation provides cumulative anti-photoaging effects that complement the acute photoprotective benefits. Chronic use benefits include enhanced baseline antioxidant status, improved DNA repair capacity, strengthened skin barrier function, increased dermal collagen density, enhanced hydration through hyaluronic acid supplementation, and reduced cumulative oxidative damage from incidental UV exposure.

Dual-use regimens may combine both acute and chronic administration strategies. In a dual-use regimen, subjects consume the composition daily for baseline photoprotection and anti-aging benefits, and may consume an additional serving 1-3 hours before planned extended UV exposure for enhanced acute protection. For example, a subject may take one serving each morning as part of a daily supplement routine, and take a second serving 2 hours before going to the beach on weekends, thereby achieving both chronic anti-aging benefits and enhanced acute photoprotection during high-exposure events.

### Aspects.

The following aspects are provided, the numbering of which is not to be construed as designating levels of importance

Aspect 1. A composition comprising: niacinamide; and polypodium leucotomos extract

Aspect 2. The composition of Aspect 1, wherein the niacinamide is present in a range of from about 25 wt % to about 45 wt % of the composition

Aspect 3. The composition of any of Aspects 1 or 2, wherein the niacinamide is present in a range of from about 35 wt % to about 40 wt % of the composition

Aspect 4. The composition of any of Aspects 1-3, wherein the niacinamide is present in a range of from about 100 mg to about 3000 mg

Aspect 5. The composition of any of Aspects 1-4, wherein the niacinamide is present in a range of from about 500 mg to about 1000 mg

Aspect 6. The composition of any of Aspects 1-5, wherein the polypodium leucotomos extract is present in a range of from about 5 wt % to about 20 wt % of the composition

Aspect 7. The composition of any of Aspects 1-6, wherein the polypodium leucotomos extract is present in a range of from about 6 wt % to about 15 wt % of the composition

Aspect 8. The composition of any of Aspects 1-7, wherein the polypodium leucotomos extract is present in a range of from about 50 mg to about 1500 mg

Aspect 9. The composition of any of Aspects 1-8, wherein the polypodium leucotomos extract is present in a range of from about 230 mg to about 350 mg

Aspect 10. The composition of any of Aspects 1-9, further comprising a polyhydroxy compound

Aspect 11. The composition of Aspect 10, wherein the polyhydroxy compound is present in a range of from about 5 wt % to about 20 wt % of the composition.

Aspect 12. The composition of any of Aspects 10 or 11, wherein the polyhydroxy compound is present in a range of from about 7 wt % to about 15 wt % of the composition

Aspect 13. The composition of any of Aspects 10-12, wherein the polyhydroxy compound is present in a range of from about 100 mg to about 1000 mg

Aspect 14. The composition of any of Aspects 10-13, wherein the polyhydroxy compound is present in a range of from about 240 mg to about 280 mg

Aspect 15. The composition of any of Aspects 10-14, wherein the polyhydroxy compound comprises ascorbic acid

Aspect 16. The composition of any of Aspects 1-15, further comprising a heterocyclic compound

Aspect 17. The composition of Aspect 16, wherein the heterocyclic compound is present in a range of from about 0.05 wt % to about 5 wt % of the composition

Aspect 18. The composition of any of Aspects 16 or 17, wherein the heterocyclic compound is present in a range of from about 0.09 wt % to about 2 wt % of the composition

Aspect 19. The composition of any of Aspects 16-18, wherein the heterocyclic compound is present in a range of from about 0.50 mg to about 10 mg

Aspect 20. The composition of any of Aspects 16-19, wherein the heterocyclic compound is present in a range of from about 3 mg to about 7 mg

Aspect 21. The composition of any of Aspects 16-20, wherein the heterocyclic compound comprises biotin

Aspect 22. The composition of any of Aspects 1-21, further comprising a carotenoid compound.

Aspect 23. The composition of Aspect 22, wherein the carotenoid compound is present in a range of from about 0.0005 wt % to about 1 wt % of the composition

Aspect 24. The composition of any of Aspects 22 or 23, wherein the carotenoid compound is present in a range of from about 0.009 wt % to about 0.005 wt % of the composition

Aspect 25. The composition of any of Aspects 22-24, wherein the carotenoid compound is present in a range of from about 0.5 mg to about 20 mg

Aspect 26. The composition of any of Aspects 22-25, wherein the carotenoid compound is present in a range of from about 2 mg to about 4 mg

Aspect 27. The composition of any of Aspects 22-26, wherein the carotenoid compound comprises astaxanthin

Aspect 28. The composition of any of Aspects 1-27, further comprising a polypeptide compound

Aspect 29. The composition of Aspect 28, wherein the polypeptide compound is present in a range of from about 0.5 wt % to about 5 wt % of the composition

Aspect 30. The composition of any of Aspects 28 or 29, wherein the polypeptide compound is present in a range of from about 2 wt % to about 4 wt % of the composition

Aspect 31. The composition of any of Aspects 28-30, wherein the polypeptide compound is present in a range of from about 10 mg to about 300 mg.

Aspect 32. The composition of any of Aspects 28-31, wherein the polypeptide compound is present in a range of from about 80 mg to about 120 mg

Aspect 33. The composition of any of Aspects 28-32, wherein the polypeptide compound comprises a bovine collagen peptide

Aspect 34. The composition of any of Aspects 1-33, further comprising a tripeptide compound

Aspect 35. The composition of Aspect 34, wherein the tripeptide compound is present in a range of from about 0.005 wt % to about 0.05 wt % of the composition

Aspect 36. The composition of any of Aspects 34 or 35, wherein the tripeptide compound is present in a range of from about 0.009 wt % to about 0.03 wt % of the composition

Aspect 37. The composition of any of Aspects 34-36, wherein the tripeptide compound is present in a range of from about 10 mg to about 150 mg

Aspect 38. The composition of any of Aspects 34-37, wherein the tripeptide compound is present in a range of from about 40 mg to about 60 mg

Aspect 39. The composition of any of Aspects 34-38, wherein the tripeptide compound comprises L-glutathione

Aspect 40. The composition of any of Aspects 1-39, further comprising a polysaccharide compound.

Aspect 41. The composition of Aspect 40, wherein the polysaccharide compound is present in a range of from about 0.005 wt % to about 0.05 wt % of the composition.

Aspect 42. The composition of any of Aspects 40 or 41, wherein the polysaccharide compound is present in a range of from about 0.008 wt % to about 0.02 wt % of the composition.

Aspect 43. The composition of any of Aspects 40-42, wherein the polysaccharide compound is present in a range of from about 5 mg to about 100 mg.

Aspect 44. The composition of any of Aspects 40-43, wherein the polysaccharide compound is present in a range of from about 20 mg to about 30 mg.

Aspect 45. The composition of any of Aspects 40-44, wherein the polysaccharide compound comprises hyaluronic acid.

Aspect 46. The composition of any of Aspects 1-45, further comprising a polyol compound.

Aspect 47. The composition of Aspect 46, wherein the polyol compound is present in a range of from about 30 wt % to about 45 wt % of the composition.

Aspect 48. The composition of any of Aspects 46 or 47, wherein the polyol compound is present in a range of from about 35 wt % to about 40 wt % of the composition.

Aspect 49. The composition of any of Aspects 46-48, wherein the polyol compound is present in a range of from about 200 mg to about 3000 mg.

Aspect 50. The composition of any of Aspects 46-49, wherein the polyol compound is present in a range of from about 950 mg to about 1050 mg.

Aspect 51. The composition of any of Aspects 46-50, wherein the polyol compound comprises inositol.

Aspect 52. The composition of any of Aspects 1-51, comprising one or more natural flavorings.

Aspect 53. The composition of any of Aspects 1-52, comprising at least one freshness preservation agent.

Aspect 54. The composition of any of Aspects 1-53, further comprising at least one freshness preservation agent selected from potassium benzoate, β-cyclodextrin, γ-cyclodextrin, or a combination thereof.

Aspect 55. A composition comprising: ascorbic acid in a range of from about 7 wt % to about 15 wt % of the composition; optionally biotin in a range of from about 0.09 wt% to about 2 wt % of the composition; niacinamide in a range of from about 35 wt % to about 40 wt % of the composition; astaxanthin in a range of from about 0.0005 wt % to about 1 wt % of the composition; bovine collagen peptides in a range of from about 2 wt % to about 4 wt % of the composition; L-glutathione in a range of from about 0.009 wt % to about 0.03 wt % of the composition; hyaluronic acid in a range of from about 0.008 wt % to about 0.02 wt % of the composition; inositol in a range of from about 35 wt % to about 40 wt % of the composition; and polypodium leucotomos extract in a range of from about 6 wt % to about 15 wt % of the composition.

Aspect 56. The composition of any of Aspects 1-55, wherein the composition is in the form of a powder, in the form of a gummy, in the form of a pill, in the form of a baked good, capsule, tablet, beverage, dry beverage mix, solution, suspension, or a combination thereof.

Aspect 57. A method comprising administering the composition of any of Aspects 1-56 to a human.

Aspect 58. The method of Aspect 57, comprising administering 1 to 5 servings of the composition to a human per day.

Aspect 59. The method of any of Aspects 57 or 58, further comprising at least partially dissolving the composition in a liquid prior to administration.

Aspect 60. The method of any of Aspects 57-59, wherein the composition reduces the incidence or severity of sun damage, sunburn, or both relative to a subject who does not receive the composition.

Aspect 61. The method of Aspect 60, wherein sun damage comprises skin cancer, damage from free radicals, environmental aging, or a combination thereof.

Aspect 62. A method of increasing minimal erythema dose (MED) in a subject comprising: administering to the subject the composition of any of Aspects 1-56.

Aspect 63. The method of Aspect 62, wherein the MED is increased by at least 30% compared to baseline measurements taken before administration to the subject.

Aspect 64. The method of Aspect 62, wherein the MED is increased by at least 10% compared to baseline.

Aspect 65. The method of Aspect 62, wherein the MED is increased by at least 60% compared to baseline.

Aspect 66. A method of reducing UV-induced erythema comprising: (a) orally administering to a subject the composition of any of Aspects 1-56; (b) waiting a period of time ranging from about 30 minutes to about 4 hours; and (c) exposing the subject to ultraviolet radiation, wherein the erythema severity at 8 hours post-UV exposure is reduced by at least 30% compared to UV exposure without prior administration of the composition

Aspect 67. The method of Aspect 66, wherein the period of time in step (b) is about 1 hour to about 3 hours

Aspect 68. The method of Aspect 66, wherein the period of time in step (b) is about 2 hours

Aspect 69. The method of Aspect 66, wherein the erythema severity is reduced by at least 10%

Aspect 70. The method of Aspect 66, wherein the erythema severity is reduced by at least 60%

Aspect 71. The method of Aspect 66, wherein the erythema severity at 24 hours post-UV exposure is reduced by at least 60% compared to UV exposure without prior administration of the composition

Aspect 72. The method of Aspect 66, wherein the erythema severity at 24 hours post-UV exposure is reduced by at least 65% compared to UV exposure without prior administration of the composition

Aspect 73. The method of Aspect 66, wherein the ultraviolet radiation comprises narrowband UVB at a wavelength of about 308 nm

Aspect 74. The method of Aspect 66, wherein the ultraviolet radiation comprises a dose of about 250 mJ/cm² to about 350 mJ/cm²

Aspect 75. The method of Aspect 66, wherein erythema severity is assessed using a standardized minimal erythema dose (MED) grading scale

Aspect 76. A composition comprising: niacinamide present in an amount of about 1000 mg; polypodium leucotomos extract present in an amount of about 240 mg; astaxanthin present in an amount of about 4 mg; optionally biotin present in an amount of about 5 mg; bovine collagen peptides present in an amount of about 100 mg; L-glutathione present in an amount of about 50 mg; hyaluronic acid present in an amount of about 25 mg; inositol present in an amount of about 1000 mg; and ascorbic acid present in an amount of about 250 mg, wherein the composition has a total weight of about 4.5 g

Aspect 77. The composition of Aspect 76, formulated as a powder for reconstitution in a liquid.

Aspect 78. The composition of Aspect 77, wherein the powder exhibits a mixability score of at least 8/10, a turbidity score of at least 8/10, a clumping score of at least 9/10, and a taste score of at least 9/10.

Aspect 79. The composition of Aspect 76, wherein the ratio of niacinamide to polypodium leucotomos extract is about 4:1 to about 4.5:1 by weight.

Aspect 80. A composition according to any of Aspects 1-56 or 76-79, wherein the composition is substantially free of turmeric.

Aspect 81. A composition according to any of Aspects 1-56 or 76-79, wherein the composition is substantially free of curcumin.

Aspect 82. A composition according to any of Aspects 1-56 or 76-79, wherein the composition is substantially free of basil extract.

Aspect 83. A composition according to any of Aspects 1-56 or 76-79, wherein the composition is substantially free of resveratrol.

Aspect 84. A composition according to any of Aspects 1-56 or 76-79, wherein the composition is substantially free of turmeric, curcumin, basil extract, and resveratrol.

Aspect 85. The composition of any of Aspects 1-56 or 76-84, wherein the polypodium leucotomos extract is present in an amount of about 200 mg to about 300 mg.

Aspect 86. The composition of any of Aspects 1-56 or 76-85, wherein the polypodium leucotomos extract is present in an amount that does not exceed 350 mg.

Aspect 87. The composition of any of Aspects 1-56 or 76-86, wherein the bovine collagen peptides are present in an amount of about 80 mg to about 150 mg.

Aspect 88. The composition of any of Aspects 1-56 or 76-87, wherein the bovine collagen peptides are present in an amount that does not exceed 200 mg.

Aspect 89. The composition of any of Aspects 1-56 or 76-88, wherein the astaxanthin is present in an amount of about 3 mg to about 5 mg.

Aspect 90. The composition of any of Aspects 1-56 or 76-89, wherein the astaxanthin is present in an amount that does not exceed 6 mg.

Aspect 91. A method of providing photoprotection to a subject comprising:
administering the composition of any of Aspects 1-56 or 76-90 to the subject once daily.
Aspect 92. The method of Aspect 91, wherein the composition is administered in the morning, approximately 1 to 2 hours before anticipated sun exposure.
Aspect 93. The method of Aspect 91 or 92, wherein photoprotective effects persist for at least 8 hours following administration.
Aspect 94. The method of any of Aspects 91-93, wherein photoprotective effects persist for at least 24 hours following administration.
Aspect 95. A method of reducing acute erythema response to controlled UV exposure comprising: administering to a subject the composition of any of Aspects 1-56 or 76-90, wherein the acute erythema response measured at 8 hours post-UV exposure is reduced compared to UV exposure without prior administration of the composition.

Aspect 96. The method of Aspect 95, wherein the subject has a Fitzpatrick skin phototype of II, III, or IV.

Aspect 97. The method of Aspect 95 or 96, wherein the reduction in acute erythema response is independent of the subject's age.

Aspect 98. The method of any of Aspects 95-97, wherein the reduction in acute erythema response is independent of the subject's sex.

Aspect 99. A method of formulating a photoprotective oral composition comprising: combining niacinamide, polypodium leucotomos extract, astaxanthin, optionally biotin, bovine collagen peptides, L-glutathione, hyaluronic acid, inositol, and ascorbic acid in amounts as defined in any of Aspects 1-56 or 76-90, while excluding turmeric in an amount greater than 1 wt % of the composition.

Aspect 100. The method of Aspect 99, further comprising evaluating the formulation for mixability, turbidity, clumping tendency, and taste to achieve a composite formulation score of at least 35/40.

Aspect 101. Use of a composition according to any of Aspects 1-56 or 76-90 for the manufacture of a medicament for reducing UV-induced skin damage.

Aspect 102. The use of Aspect 101, wherein the UV-induced skin damage comprises erythema, sunburn, DNA damage, oxidative stress, inflammation, or combinations thereof.

Aspect 103. A kit comprising: (a) the composition of any of Aspects 1-56 or 76-90; and (b) instructions for use directing administration of the composition approximately 1 to 2 hours prior to anticipated UV exposure.

Aspect 104. The kit of Aspect 103, further comprising a measuring device for accurate dosing.

Aspect 105. The kit of Aspect 103 or 104, wherein the composition is packaged in individual serving packets.

Aspect 106. A composition according to any of Aspects 1-56 or 76-90 for use in reducing the severity of sunburn in a subject exposed to ultraviolet radiation.

Aspect 107. A composition according to any of Aspects 1-56 or 76-90 for use in increasing the minimal erythema dose (MED) in a subject.

Aspect 108. A composition according to any of Aspects 1-56 or 76-90 for use in providing acute photoprotection within 1 to 3 hours of oral administration.

Aspect 109. The composition, method, kit, or use of any one or any combination of Aspects 1-108, optionally configured such that all elements or options recited are available to use or select from.

What is claimed is:

1. A composition comprising: niacinamide; and polypodium leucotomos extract.

2. The composition of claim 1, wherein the niacinamide is present in a range of from about 35 wt % to about 40 wt % of the composition.

3. The composition of claim 1, wherein the polypodium leucotomos extract is present in a range of from about 6 wt % to about 15 wt % of the composition.

4. The composition of claim 1, further comprising ascorbic acid.

5. The composition of claim 4, further comprising biotin.

6. The composition of claim 5, further comprising astaxanthin.

7. The composition of claim 6, further comprising a bovine collagen peptide.

8. The composition of claim 7, further comprising L-glutathione.

9. The composition of claim 8, further comprising hyaluronic acid.

10. The composition of claim 9, further comprising inositol.

11. A composition comprising: ascorbic acid in a range of from about 7 wt % to about 15 wt % of the composition; optionally biotin in a range of from about 0.09 wt % to about 2 wt % of the composition; niacinamide in a range of from about 35 wt % to about 40 wt % of the composition; astaxanthin in a range of from about 0.0005 wt % to about 1 wt % of the composition; bovine collagen peptides in a range of from about 2 wt % to about 4 wt % of the composition; L-glutathione in a range of from about 0.009 wt % to about 0.03 wt % of the composition; hyaluronic acid in a range of from about 0.008 wt % to about 0.02 wt % of the composition; inositol in a range of from about 35 wt % to about 40 wt % of the composition; and polypodium leucotomos extract in a range of from about 6 wt % to about 15 wt % of the composition.

12. The composition of claim 11, wherein the composition is in the form of a powder, gummy, capsule, tablet, beverage, or dry beverage mix.

13. A composition comprising: niacinamide present in an amount of about 1000 mg; polypodium leucotomos extract present in an amount of about 240 mg; astaxanthin present in an amount of about 4 mg; optionally biotin present in an amount of about 5 mg; bovine collagen peptides present in an amount of about 100 mg; L-glutathione present in an amount of about 50 mg;
hyaluronic acid present in an amount of about 25 mg; inositol present in an amount of about 1000 mg; and ascorbic acid present in an amount of about 250 mg, wherein the composition has a total weight of about 4.5 g.

14. The composition of claim 13, wherein the composition is substantially free of turmeric, curcumin, basil extract, and resveratrol.

15. A method of increasing minimal erythema dose (MED) in a subject comprising:
administering to the subject the composition of claim 13.

16. The method of claim 15, wherein the MED is increased by at least 10% compared to baseline.

17. A method of reducing UV-induced erythema comprising: (a) orally administering to a subject the composition of claim 13; (b) waiting a period of time ranging from about 30 minutes to about 4 hours; and (c) exposing the subject to ultraviolet radiation, wherein the erythema severity at 8 hours post-UV exposure is reduced by at least 30% compared to UV exposure without prior administration of the composition.

18. The method of claim 17, wherein the erythema severity is reduced by at least 10%.

19. A kit comprising: (a) the composition of claim 13; and (b) instructions for use directing administration of the composition approximately 1 to 2 hours prior to anticipated UV exposure.

20. The composition of claim 13 for use in increasing the minimal erythema dose (MED) in a subject.
